(19) United States
(12) Patent Application Publication
(10) Pub. No.: US 2026/0174518 A1
(43) Pub. Date: Jun. 25, 2026

(54) FORCE SENSING MEDICAL INSTRUMENT

(71) Applicant: Intuitive Surgical Operations, Inc., Sunnyvale, CA (US)

(72) Inventors: Lizmarie COMENENCIA ORTIZ, San Bruno, CA (US); David I. MOREIRA RIDSDALE, Saratoga, CA (US); Ashwinram SURESH, San Jose, CA (US); Cheng-Ling CHANG, Davis, CA (US)

(73) Assignee: Intuitive Surgical Operations, Inc., Sunnyvale, CA (US)

(21) Appl. No.: 19/129,121

(22) PCT Filed: Nov. 13, 2023
(86) PCT No.: PCT/US2023/079517

§ 371 Date: May 12, 2025

Related U.S. Application Data
(60) Provisional application No. 63/425,518, filed on Nov. 15, 2022.

(51) Int. Cl. A61B 34/35; A61B 90/00; G01L 1/20; G01L 1/22

(52) U.S. Cl. CPC A61B 34/35; A61B 90/06; G01L 1/205; G01L 1/2262; G01L 1/2268; G01L 1/2287; A61B 2090/064

(57) ABSTRACT

Systems and methods are provided for control of a surgical system. A force sensing instrument for use with the surgical system includes a force sensor unit. The force sensor unit is configured to mitigate electromagnetic interference with an output signal. Accordingly, the force sensor unit includes an electrically conductive layer that is over a lateral surface of a resiliently deflectable beam. An electrically insulative layer is over the electrically conductive layer. A strain sensor is over the electrically insulative layer. The electrically conductive layer is mechanically bonded along its length to the beam. The electrically conductive layer is also electrically coupled to the beam.

### CROSS-REFERENCE TO RELATED APPLICATIONS

This application claims priority to and the filing date benefit of U.S. Provisional Patent Application No. 63/425,518, entitled “Force Sensing Medical Instrument,” filed Nov. 15, 2022, the disclosure of which is incorporated herein by reference in its entirety.

### BACKGROUND

The embodiments described herein relate to force sensing technology, and more specifically to force sensing technology adapted for use with teleoperated surgical systems. More particularly, the embodiments described herein relate to force sensing medical instruments for determining forces applied to the medical instrument in order to control a surgical system that includes a force feedback that may be provided to a system operator. Still more particularly, the embodiments described herein relate to the mitigation of electromagnetic interference when the force sensing medical instrument is exposed to an electrical field.

Known techniques for Minimally Invasive Surgery (MIS) employ instruments to manipulate tissue that can be either manually controlled or controlled via hand-held or mechanically grounded teleoperated medical systems that operate with at least partial computer-assistance (“telesurgical systems”). Many known MIS instruments include a therapeutic or diagnostic end effector (e.g., forceps, a cutting tool, or a cauterizing tool) mounted on an optional wrist mechanism at the distal end of a shaft. During an MIS procedure, the end effector, wrist mechanism, and the distal end of the shaft are typically inserted into a small incision or a natural orifice of a patient via a cannula to position the end effector at a work site within the patient's body. The optional wrist mechanism can be used to change the end effector's position and orientation with reference to the shaft to perform a desired procedure at the work site. In known instruments, motion of the instrument as a whole provides mechanical degrees of freedom (DOFs) for movement of the end effector and the wrist mechanisms generally provide the desired DOFs for movement of the end effector with reference to the shaft of the instrument. For example, for forceps or other grasping tools, known wrist mechanisms are able to change the pitch and yaw of the end effector with reference to the shaft. A wrist may optionally provide a roll DOF for the end effector, or the roll DOF may be implemented by rolling the shaft. An end effector may optionally have additional mechanical DOFs, such as grip or knife blade motion. In some instances, wrist and end effector mechanical DOFs may be combined. For example, U.S. Pat. No. 5,792,135 (filed May 16, 1997) discloses a mechanism in which wrist and end effector grip DOFs are combined.

Force sensing medical instruments are known and, together with associated telesurgical systems, may deliver haptic feedback during a MIS procedure to a surgeon performing the procedure. The haptic feedback may increase the immersion, realism, and intuitiveness of the procedure. For effective haptics rendering and accuracy, force sensors may be placed on a medical instrument and as close to the anatomical tissue interaction as possible. One approach is to include a force sensor unit having electrical sensor elements (e.g., strain sensors or strain gauges) at a distal end of a medical instrument shaft to measure strain imparted to the medical instrument. The measured strain can be used to determine the force imparted to the medical instrument and as input upon which the desired haptic feedback may be generated.

In some MIS procedures an electrical current is introduced to the surgical site, such as during electrosurgery. Electrosurgery refers broadly to a class of medical procedures that rely on the application of high frequency electrical energy, usually radio frequency energy, to patient tissue to achieve a number of possible effects, such as cutting, coagulation, necrosis, and the like. For example, in some MIS procedures tissue in the patient's body must be cauterized and severed. To perform such a procedure, end effector grips configured to apply bipolar or monopolar cauterizing energy are introduced to the surgical site to engage the target tissue, and electrical energy, such as radiofrequency energy, is delivered to the grips to cauterize the engaged tissue. Alternatively, in some instances surgeons have been known to engage tissue with electrically conductive end effector grips that are not specifically configured to apply electrical energy, and then place an actively charged electrode (such as an electrically charged end effector on a second instrument) in electrically conductive contact (i.e., direct electrical coupling) with the grips in order to apply electrosurgical energy to the tissue.

Force sensing instruments may be specifically designed to apply electrosurgical energy (e.g., a bipolar forceps instrument) or not designed to apply electrosurgical energy (e.g., a Cadiere forceps instrument). Regardless of whether a force sensing medical instrument is designed to apply electrosurgical energy, during certain MIS procedures, the force sensing medical instrument can be exposed to an electrical field during an electrosurgical operation. And regardless of the approach used to apply electrosurgical energy to tissue—with an instrument specifically designed to apply electrosurgical energy or with an instrument not specifically designed to apply electrosurgical energy—electrical current associated with the electrosurgical energy can be conducted through or along various components of the force sensing medical instrument.

The exposure of the force sensing medical instrument to the electrical field can result in the generation of electromagnetic interference within the instrument that can affect signals from the force sensing instrument's force sensor unit. In turn, this effect on the signals can result in inaccurate indications of the forces acting on the force sensing medical instrument and the associated haptic feedback to the surgeon operating the force sensing instrument. Insofar as the haptic feedback is based on the indications of force on the instrument, it is desirable to mitigate the effects of the electromagnetic interference. Such mitigations are subject to the design and design constraints (e.g., component materials needed for strength or other mechanical properties, small component sizes required for surgery, etc.) of the force sensing instrument itself. For example, one approach has attempted to employ a Faraday cage around any components that could be affected. This required additional conductive components enclosing the entirety of the sensor along the complete instrument length, an effective grounding of the cage, and additional clearance. However, due to the additional components, this approach could adversely affect sensor performance (e.g., alignment, calibration, and/or robustness).

The magnitude and/or affect of the electromagnetic interference on the output of the force sensor unit can also depend, at least in part, on the positioning of various components of the force sensing medical instrument. For example, the conductive contact between the electrode and the force sensing medical instrument with an instrument not specifically designed to apply electrosurgical energy can result in the electrical current being conducted via a conductive component (e.g., a metal component such as a beam, a mechanical cable, and/or a shaft) of the force sensing medical instrument. The electrically conductive component can be separated from another electrically conductive component (e.g., strain sensors, strain gauges, and/or sensor cables) of the force sensing medical instrument by electrical insulation. However, the two electrically conductive components can become capacitively or inductively coupled (i.e., indirectly electrically coupled) when the current in the first component generates a current through the insulation into the second component. The magnitude of the generated current is affected, at least in part, by the positioning of the two conductive components and by the insulation therebetween. For example, a strain sensor can be mechanically coupled to an electrically conductive structure by an electrically insulative adhesive. In accordance with the principles of capacitive coupling, a current conducted by the structure can generate a current in the strain sensor through the electrically insulative adhesive. The magnitude of the generated current can be affected by a distance between the strain sensor and the structure as determined by the thickness of the electrically insulative adhesive and other factors. Insofar as changes in the relatively low voltage of the strain sensor can be indicative of the forces acting on the force sensing medical instrument, the presence of electromagnetic interference (in the form of the generated current) in the output of the strain sensor can distort the force indications.

In addition to the capacitive coupling, electromagnetic interference can also result from the inductive coupling (e.g., antenna coupling or magnetic field coupling) of various components of the force sensing medical instrument. When inductively coupled, a magnetic field resulting from an electrical current in one conductor generates an electrical current in a second conductor. For example, a current can be generated via inductive coupling in a portion of the strain sensor and/or the sensor cable carrying signals from the strain sensor. The presence of the current generated by the inductive coupling is electromagnetic interference that can distort the indications of strain generated by the force sensor unit, resulting in discrepancies in the indications of the force acting on the force sensing medical instrument.

In view of the aforementioned, the art is continuously seeking new and improved systems and methods for control of a surgical system based on the accurate measurement of the strain imparted to the medical instrument.

### SUMMARY

This summary introduces certain aspects of the embodiments described herein to provide a basic understanding. This summary is not an extensive overview of the inventive subject matter, and it is not intended to identify key or critical elements or to delineate the scope of the inventive subject matter.

The systems and methods described herein facilitate the control of a surgical system when the force sensor unit of a force sensing medical instrument is exposed to an electrical field. In particular the force sensor unit is configured to mitigate the effects of electromagnetic interference. With the electromagnetic effects being mitigated, the force sensor unit can output strain signals that accurately indicate the forces affecting the force sensing medical instrument.

In one aspect, the present disclosure is directed to a force sensor unit. The force sensor unit can be employed in a force sensing medical instrument (“instrument”). The instrument can, for example, be used with a surgical system in the performance of a minimally invasive surgery. The force sensor unit includes a beam that has a lateral surface. The beam is configured to deflect in response to a force affecting a distal end (e.g., a tool member) of the instrument. An electrically conductive layer is over the lateral surface. An electrically insulative layer over the electrically conductive layer. A strain sensor is over a length of the electrically insulative layer. The strain sensor is configured to output a strain indication in response to the deflection of the beam. The electrically conductive layer is mechanically bonded to the lateral surface of the beam along a length of the electrically conductive layer, and the electrically conductive layer is electrically coupled to the beam.

In some embodiments, the electrically conductive layer has a top surface facing the strain sensor. The top surface has a flatness that is within a specified flatness tolerance. In some embodiments, the specified flatness tolerance is 0.1 micrometers or less. In some embodiments, the electrically conductive layer has a surface roughness of less than 0.1 micrometers.

In some embodiments, the electrically conductive layer includes two or more weld locations. The electrically conductive layer is electrically coupled to the beam at each of the weld locations. In some embodiments, the two or more weld locations are positioned to produce a spatially uniform electrical coupling of the electrically conductive layer to the beam.

In some embodiments, the electrically conductive layer is a stainless steel, and the electrically insulative layer is a polyimide film.

In some embodiments, the electrically conductive layer is mechanically bonded to the lateral surface of the beam along the length of the electrically conductive layer via an adhesive. The adhesive is distributed laterally across the lateral surface of the beam and longitudinally along the lateral surface of the beam. The adhesive covers at least 95 percent of a surface of the electrically conductive layer facing the beam.

In some embodiments, the electrically insulative layer has a uniform thickness. The uniform thickness establishes a uniform separation distance between the strain sensor and the electrically conductive layer. In some embodiments, the electrically conductive layer has a thickness that is within a specified thickness range. The specified thickness range is greater than 45 micrometers and less than 55 micrometers.

In some embodiments, the strain sensor includes a bridge circuit, a set of electrical pads, and an electrical trace structure. The strain sensor has a stiffness extending parallel to the lateral surface of the beam. The bridge circuit includes a set of strain gauges formed over the electrically insulative layer. The bridge circuit has a uniform separation distance from the electrically conductive layer. The electrical trace structure is electrically coupled to the set of electrical pads. The set of strain gauges is positioned on a gauge plane that is parallel to the electrically conductive layer. The electrical trace structure is positioned on a trace plane that is parallel to the gauge plane. The electrical trace structure is laterally offset from each strain gauge of the set of strain gauges to facilitate a uniformity in the stiffness of the strain sensor.

In some embodiments, the strain sensor has a stiffness extending parallel to the lateral surface. The force sensor unit includes an enclosure layer, and the enclosure layer covers the strain sensor. The enclosure layer has a uniform thickness that facilitates a uniformity in the stiffness of the strain sensor.

In some embodiments, the strain sensor includes a bridge circuit. The bridge circuit includes a plurality of strain gauges formed over the electrically insulative layer. A wall surrounds the bridge circuit and has a height that is equal to or greater than a thickness of the strain gauges.

In an additional aspect, the present disclosure is directed to additional embodiments of a force sensor unit. The force sensor unit includes a beam that has a lateral surface. The beam is configured to deflect in response to a force affecting a distal end (e.g., a tool member) of the instrument. A strain sensor is mechanically coupled to the beam. The strain sensor is configured to output a strain indication in response to the deflection of the beam. The strain sensor includes a first region, a second region, a bridge circuit, an electrical trace structure, and a balancing structure. The electrical trace structure is over the first region and the balancing structure is over the second region to maintain symmetry and uniformity of the strain sensor. The electrical trace structure is electrically coupled to the bridge circuit, while the balancing structure has an absence of physical electrical connections with any other component of the force sensor unit.

In some embodiments, the electrical trace structure includes a first area portion, and the balancing structure includes a second area portion. An outline of the first area portion of the electrical trace structure defines a first pattern having a first surface area, and an outline of the second area portion of the balancing structure defines a second pattern having a second surface area. The second pattern of the balancing structure matches the first pattern of the electrical trace structure, and the second surface area of the balancing structure equals the first surface area. In some embodiments, the second pattern of the balancing structure is configured to generate a first voltage change in the strain sensor that is proportional to a second voltage change generated in the strain sensor by the electrical trace structure.

In some embodiments, the electrical trace structure includes an input trace separated from one or more measurement traces. The first area portion of the electrical trace structure includes a portion of the input trace and a portion of the one or more measurement traces. The second pattern defines a void that corresponds to the separation between the input trace and the one or more measurement traces.

In some embodiments, a longitudinal axis of the second pattern of balancing structure is aligned with a longitudinal axis of the first pattern of the electrical trace structure.

In some embodiments, the force sensor unit includes an electrically conductive layer and an electrically insulative layer. The beam includes a lateral surface, and the electrically conductive layer is over the lateral surface. The electrically insulative layer is over the electrically conductive layer. The strain sensor is over a length of the electrically insulative layer. The electrically conductive layer is mechanically bonded to the lateral surface of the beam along the length of the electrically conductive layer. The electrically conductive layer is electrically coupled to the beam.

In some embodiments, the strain sensor has a stiffness extending parallel to the lateral surface of the beam. The bridge circuit is one of eight half-bridge circuits. Each of the 8 half-bridge circuits includes a set of strain gauges. The eight half-bridge circuits are arranged as four full-bridge-circuit combinations (e.g., four pairings of electrically coupled half-bridge circuits). The electrical trace structure is a first electrical trace structure of four electrical trace structures. Each of the four electrical trace structures includes an input trace separated from one or more measurement traces. The balancing structure is one of four balancing structures. Each bridge circuit includes two strain gauges of the set of strain gauges formed over the electrically insulative layer. Each of the strain gauges is positioned within a gauge plane that is parallel to the electrically conductive layer and has a uniform separation distance from the electrically conductive layer. Each of the four electrical trace structures and each of the four balancing structures is positioned within a lead plane that is over and parallel to the gauge plane. Each of the four electrical trace structures and each of the four balancing structures is laterally offset from each strain gauge of the set of strain gauges to facilitate a uniformity in the stiffness of the strain sensor.

In some embodiments, the beam includes a beam center axis. The four full-bridge-circuit arrangement include a primary proximal bridge-circuit combination, a primary distal bridge-circuit combination, a secondary proximal bridge-circuit combination, and a secondary distal bridge-circuit combination. The primary proximal bridge-circuit combination is proximal of the secondary proximal bridge-circuit combination. The primary distal bridge-circuit combination is proximal of the secondary distal bridge-circuit combination and distal of the secondary proximal bridge-circuit combination. A first electrical trace structure and a second electrical trace structure of the four electrical trace structures are positioned on opposite sides of the beam center axis, equidistant from the beam center axis, and adjacent to the secondary distal bridge-circuit combination. A third electrical trace structure and a fourth electrical trace structure of the four electrical trace structures are positioned on opposite sides of the beam center axis, equidistant from the beam center axis, and adjacent to the primary proximal bridge-circuit combination. A first balancing structure of the four balancing structures is positioned proximal to the first electrical trace structure, in alignment with the first electrical trace structure, and adjacent to the primary distal bridge-circuit combination. A second balancing structure is positioned proximal to the second electrical trace structure, in alignment with the second electrical trace structure, and adjacent to the primary distal bridge-circuit combination. A third balancing structure is positioned proximal to the third electrical trace structure, in alignment with the third electrical trace structure, and adjacent to the secondary proximal bridge-circuit combination. A fourth balancing structure is positioned proximal to the fourth electrical trace structure, in alignment with the fourth electrical trace structure, and adjacent to the secondary proximal bridge-circuit combination.

In some embodiments, the beam includes a beam center axis extending longitudinally between a distal end portion and a proximal end portion of the beam. The eight half-bridge circuits include a first half-bridge circuit, a third half-bridge circuit, a fifth half-bridge circuit, and a seventh half-bridge circuit positioned at the distal end portion of the beam. The eight half-bridge circuits also include a second half-bridge circuit, a fourth half-bridge circuit, a sixth half-bridge circuit, and an eighth half-bridge circuit positioned at the proximal end portion of the beam. The first half-bridge circuit and the second half-bridge circuit are electrically coupled to form a first primary-full-bridge circuit. The third half-bridge circuit and the fourth half-bridge circuit are electrically coupled to form a second primary-full-bridge circuit. The fifth half-bridge circuit and the sixth half-bridge circuit are electrically coupled to form a first secondary-full-bridge circuit. The seventh half-bridge circuit and the eighth half-bridge circuit are electrically coupled to form a second secondary-full-bridge circuit. A first electrical trace structure and a second electrical trace structure of the four electrical trace structures are positioned on opposite sides of the beam center axis, equidistant from the beam center axis, and adjacent to the seventh half-bridge circuit. A third electrical trace structure and a fourth electrical trace structure of the four electrical trace structures are positioned on opposite sides of the beam center axis, equidistant from the beam center axis, and adjacent to the fourth half-bridge circuit. A first balancing structure of the four balancing structures is positioned proximal to the first electrical trace structure, in alignment with the first electrical trace structure, and adjacent to the third half-bridge circuit. A second balancing structure of the four balancing structures is positioned proximally to the second electrical trace structure, in alignment with the second electrical trace structure, and adjacent to the third half-bridge circuit. A third balancing structure of the four balancing structures is positioned proximal to the third electrical trace structure, in alignment with the third electrical trace structure, and adjacent to the eighth half-bridge circuit. Additionally, a fourth balancing structure of the four balancing structures is positioned proximal to the fourth electrical trace structure, in alignment with the fourth electrical trace structure, and adjacent to the eighth half-bridge circuit.

In some embodiments, the fifth half-bridge circuit and the sixth half-bridge circuit are positioned longitudinally between the first half-bridge circuit and the second half-bridge circuit. The seventh half-bridge circuit and the eighth half-bridge circuit are positioned distally relative to the second half-bridge circuit and the fourth half-bridge circuit.

In some embodiments, the bridge circuit includes a set of strain gauges. A wall surrounds the bridge circuit and the wall has a height that is equal to or greater than a thickness of the strain gauges.

In some embodiments, the strain sensor has a stiffness extending parallel to the lateral surface. The force sensor unit includes an enclosure layer that covers the strain sensor. The enclosure layer has a uniform thickness that facilitates a uniformity in the stiffness of the strain sensor.

### BRIEF DESCRIPTION OF THE DRAWINGS

FIG. 1 is a plan view of a minimally invasive teleoperated medical system according to an embodiment being used to perform a medical procedure such as surgery.

FIG. 2 is a perspective view of a user control console of the minimally invasive teleoperated surgery system shown in FIG. 1.

FIG. 3 is a perspective view of an optional auxiliary unit of the minimally invasive teleoperated surgery system shown in FIG. 1.

FIG. 4 is a front view of a manipulator unit, including a set of instruments, of the minimally invasive teleoperated surgery system shown in FIG. 1.

FIG. 5 is a perspective view of a force sensing medical instrument with a back cover removed for clarity according to an embodiment.

FIG. 6 is a side view of a portion of the instrument of FIG. 5 with an outer shaft removed.

FIG. 7 is a schematic top view illustration of a force sensor unit according to an embodiment.

FIG. 8 is a close up of a portion of the force sensor unit of FIG. 7 depicting area portions, outlines, and patterns of an electrical trace structure and a balancing structure.

FIG. 9 is a schematic section view illustration of the force sensor unit of FIG. 7 taken at x₁-x₁.

FIG. 10 is a perspective view of a force sensor unit according to an embodiment.

FIG. 11A is an electrical schematic illustration of an example configuration of a strain sensor of the force sensor unit shown in FIG. 10.

FIG. 11B is an electrical schematic illustration of another example configuration of a strain sensor of the force sensor unit shown in FIG. 10.

FIG. 11C is a schematic illustration of an example layout of the strain gauges of the strain sensor of FIG. 11B.

FIG. 12 is a perspective view of a portion of the force sensor unit of FIG. 10 according to an embodiment.

FIG. 13 is a partial schematic side view of the portion of the force sensor unit depicted in FIG. 12.

FIG. 14 is a perspective view of a portion of the force sensor unit of FIG. 10 illustrating another alternative arrangement of the strain gauge resistors according to another embodiment.

FIG. 15 is an enlarged illustration of a portion of the force sensor unit shown in FIG. 10 illustrating yet another alternative arrangement of the strain gauge resistors according to the configuration and layout shown in FIGS. 11B-11C.

FIG. 16 is a schematic illustration of a controller for use with a minimally invasive teleoperated surgery system according to an embodiment.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

The embodiments described herein can advantageously be used in a wide variety of grasping, cutting, and manipulating operations associated with minimally invasive surgery. The medical instruments or devices of the present application enable motion in three or more degrees of freedom (DOFs). For example, in some embodiments, an end effector of the medical instrument can move with reference to the main body of the instrument in three mechanical DOFs, e.g., pitch, yaw, and roll (shaft roll). There may also be one or more mechanical DOFs in the end effector itself, e.g., two jaws, each rotating with reference to a clevis (2 DOFs) and a distal clevis that may rotate with reference to a proximal clevis (one DOF). Thus, in some embodiments, the medical instruments or devices of the present application may enable motion in six DOFs. The embodiments described herein further may be used to deliver haptic feedback to a system operator based on a load indication from the force sensor unit as modified by the force sensor bias value.

Generally, the present disclosure is directed to systems and methods for controlling a surgical system (system) such as a minimally invasive teleoperated surgery system. In particular, the present disclosure includes a force sensor unit configured to mitigate electromagnetic interference. The force sensor unit can be employed with a force sensing medical instrument (instrument) to provide an indication of force affecting the instrument. This indication of the force(s) can be used by the system to deliver haptic feedback to a user control unit of the system.

As described herein, the force sensor unit includes a strain sensor coupled to a resiliently deformable beam. The beam is configured to deform in response to a load affecting a distal end portion of the instrument. The strain sensor includes strain gauges that measure the resultant strain in the beam due to the deflection. The strain sensor indicates the strain magnitude in the form of relatively small voltage differentials. In some embodiments, the strain gauges are arranged in a split-bridge configuration (e.g., a split Wheatstone bridge) with one half of the split, full-bridge being coupled to a positive trace configured to carry a signal at a positive electrical potential and the other half being coupled to a negative trace configured to carry a signal at a negative electrical potential. The voltage differential, as opposed to an absolute voltage, between the signal carried by the positive trace and the signal carried by the negative trace is indicative of the measured strain magnitude in the absence of electromagnetic interference.

During certain procedures, the force sensor unit can be exposed to an electrical field. This exposure can result in the development of electromagnetic interference that can affect the signals in the positive and/or negative traces. For example, a current conducted through a portion of the force sensor unit, such as the beam, can induce an unintended current in another portion of the force sensor unit. The induced current can result from capacitive coupling and/or inductive coupling between the various conductive components of the force sensor unit. The magnitude of the induced current, and thus the magnitude of the electromagnetic interference, can be affected by the positions and/or orientations of the various conductive components of the force sensor unit relative to one another. When the magnitude of the electromagnetic interference (i.e., the induced current(s)) in one of the traces is greater than the magnitude of the electromagnetic interference in other trace, then the voltage differential, and thus the measured strain magnitude, is distorted. However, when a difference between the magnitude of the electromagnetic interference in each of the traces is minimized, the effect of electromagnetic interference in one trace is substantially cancelled out by the electromagnetic interference in the other trace, and vice versa. Accordingly, it is desirable to mitigate the effects of the electromagnetic interference by minimizing a differential between the induced current in the positive trace coupled to one half of the split, full-bridge and the induced current in the corresponding negative trace coupled to the other half of the full-bridge.

In some operations, exposure to the electrical field can result in an electric current being conducted by the beam. This current can induce, via capacitive coupling, a current in the strain sensor components that are mechanically coupled to the beam by an insulator. However, the distance between each of the components of the strain sensor and the beam can vary based, for example, on variability in the thickness of the adhesive employed to couple the components to the beam. This variability in the distance between the components in the beam results in capacitively induced currents of varying magnitudes within the strain sensor. Accordingly, in some embodiments the force sensor unit described herein to reduce or eliminate the variability in the magnitudes of the induced currents. The force sensor unit utilizes an electrically conductive layer positioned between the beam and the strain sensor, with an electrically insulative layer positioned between the electrically conductive layer and the strain sensor. As such, the insulative layer can have a uniform thickness and the electrically conductive layer can have a flatness that is within a specified flatness tolerance. The uniform thickness and/or the flatness can establish the strain sensor at a uniform separation distance from the electrically conductive layer. The electrically conductive layer is electrically coupled to the beam such that a current conducted by the beam is likewise conducted by the electrically conductive layer. As a result, the magnitude of the capacitively induced current in the various components (e.g., the strain gauges) of the strain sensor is determined by the uniform distance between the strain sensor and the electrically conductive element rather than by the variable distances between the strain sensor components and the beam. As the strain sensor has a uniform separation distance with the electrically conductive layer, the induced current introduced to the positive trace is substantially equal to the induced current introduced to the corresponding negative trace, resulting in the canceling out of the electromagnetic interference effects.

In some operations, exposure to the electrical field can result in electromagnetic interference resulting from inductive coupling between various components of the strain sensor. In order to mitigate the effects of the inductive coupling, the strain sensor can be configured to maximize longitudinal symmetry and lateral symmetry. The symmetry of the strain sensor facilitates the canceling out of the various inductively induced currents, and thus the canceling out of the effects of electromagnetic interference. For example, as described herein, the strain sensor can include a first region that is adjacent to a first strain gauge and a second region that is adjacent to a second strain gauge. An electrical trace structure (e.g., an input trace and a measurement trace) can be positioned over the first region. An electrically conductive balancing structure, which does not have a physical electrical connection with any component of the force sensor unit, can be positioned over the second region. In this arrangement, the electrical trace structure and the first strain gauge can be inductively coupled when the instrument is exposed to the electrical field. Similarly, the balancing structure and the second strain gauge can be inductively coupled. As such, the effects of an induced current resulting from the inductive coupling between the electrical trace structure and the first strain gauge can be counteracted by the induced current resulting from the inductive coupling between the balancing structure and a second strain gauge.

In addition to balancing the effects of inductive coupling, the balancing structure can also facilitate a stiffness uniformity of the strain gauge. In other words, the balancing structure can be employed to counteract stiffness concentrations that result from the positioning of other components of the strain sensor. The degree to which the stiffness of the strain sensor is uniform both laterally and longitudinally, can affect the accuracy of the strain sensor. For example, even the relatively insignificant difference in rigidity between a volume of constantan with a copper trace and a corresponding volume of constantan without a copper trace can affect strain indications from the strain sensor. Accordingly, the balancing structure can be positioned in a portion (e.g., the second region) of the strain sensor that would otherwise lack a copper trace in order to balance the effect on stiffness of the electrical trace structure in another portion (e.g., the first region).

As used herein, the term “about” when used in connection with a referenced numeric indication means the referenced numeric indication plus or minus up to 10 percent of that referenced numeric indication. For example, the language “about 50” covers the range of 45 to 55. Similarly, the language “about 5” covers the range of 4.5 to 5.5.

As used in this specification and the appended claims, the word “distal” refers to direction towards a work site, and the word “proximal” refers to a direction away from the work site. Thus, for example, the end of a tool that is closest to the target tissue would be the distal end of the tool, and the end opposite the distal end (i.e., the end manipulated by the user or coupled to the actuation shaft) would be the proximal end of the tool.

Further, specific words chosen to describe one or more embodiments and optional elements or features are not intended to limit the invention. For example, spatially relative terms—such as “beneath”, “below”, “lower”, “above”, “upper”, “proximal”, “distal”, and the like—may be used to describe the relationship of one element or feature to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes includes various spatial device positions and orientations. The combination of a body's position and orientation define the body's pose (e.g., a kinematic pose).

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

In addition, the singular forms “a”, “an”, and “the” are intended to include the plural forms as well, unless the context indicates otherwise. The terms “comprises”, “includes”, “has”, and the like specify the presence of stated features, steps, operations, elements, components, etc. but do not preclude the presence or addition of one or more other features, steps, operations, elements, components, or groups.

Unless indicated otherwise, the terms “apparatus,” “medical device,” “instrument,” “medical instrument,” “surgical instrument,” and variants thereof, can be interchangeably used.

Inventive aspects are described with reference to a teleoperated surgical system. An example architecture of such a teleoperated surgical system is the da Vinci® surgical system commercialized by Intuitive Surgical, Inc., Sunnyvale, California. Knowledgeable persons will understand, however, that inventive aspects disclosed herein may be embodied and implemented in various ways, including computer-assisted, non-computer-assisted, and hybrid combinations of manual and computer-assisted embodiments and implementations. Implementations are merely presented as examples, and they are not to be considered as limiting the scope of the inventive aspects disclosed herein. As applicable, inventive aspects may be embodied and implemented in both relatively smaller, hand-held, hand-operated devices and relatively larger systems that have additional mechanical support.

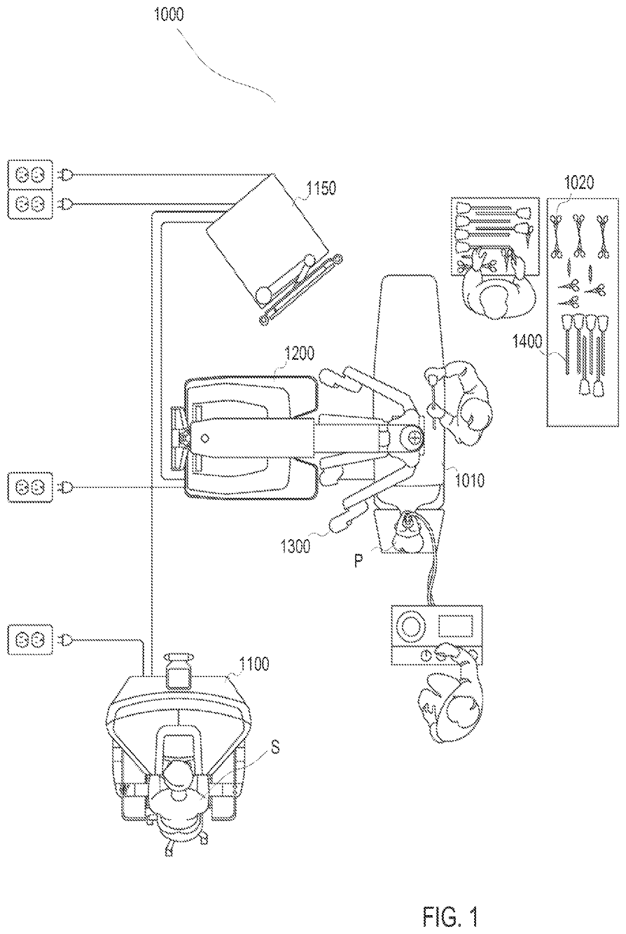

FIG. 1 is a plan view illustration of a teleoperated surgical system (“system”) 1000 that operates with at least partial computer assistance (a “telesurgical system”). Both telesurgical system 1000 and its components are considered medical devices. Telesurgical system 1000 is a Minimally Invasive Robotic Surgical (MIRS) system used for performing a minimally invasive diagnostic or surgical procedure on a Patient P who is lying on an Operating table 1010. The system can have any number of components, such as a user control unit 1100 for use by an operator of the system, such as a surgeon or other skilled clinician S, during the procedure. The MIRS system 1000 can further include a manipulator unit 1200 (popularly referred to as a surgical robot) and an optional auxiliary equipment unit 1150. The manipulator unit 1200 can include an arm assembly 1300 and a surgical instrument tool assembly removably coupled to the arm assembly. The manipulator unit 1200 can manipulate at least one removably coupled medical instrument (instrument) 1400 (e.g., a force sensing medical instrument) through a minimally invasive incision in the body or natural orifice of the patient P while the surgeon S views the surgical site and controls movement of the instrument 1400 through control unit 1100. An image of the surgical site is obtained by an endoscope (not shown), such as a stereoscopic endoscope, which can be manipulated by the manipulator unit 1200 to orient the endoscope. The auxiliary equipment unit 1150 can be used to process the images of the surgical site for subsequent display to the Surgeon S through the user control unit 1100. The number of instruments 1400 used at one time will generally depend on the diagnostic or surgical procedure and the space constraints within the operating room, among other factors. If it is necessary to change one or more of the instruments 1400 being used during a procedure, an assistant removes the instrument 1400 from the manipulator unit 1200 and replaces it with another instrument 1400 from a tray 1020 in the operating room. Although shown as being used with the instruments 1400, any of the instruments described herein can be used with the system 1000.

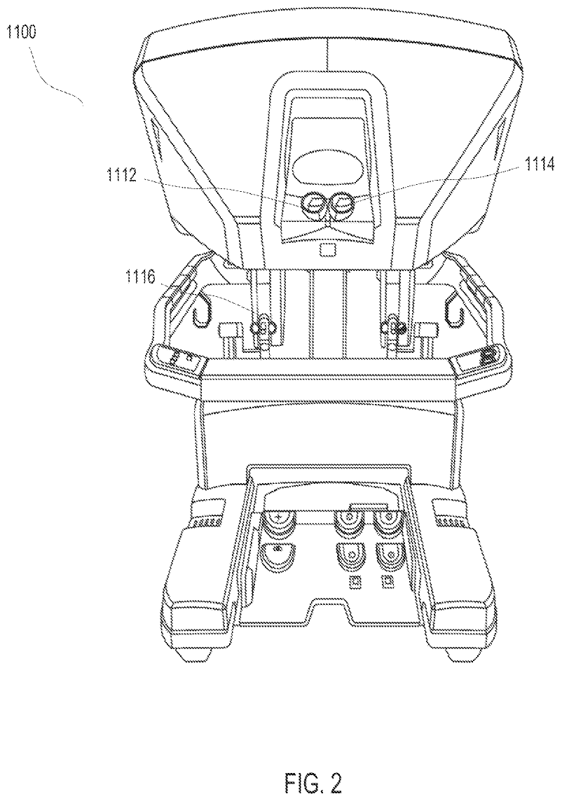

FIG. 2 is a perspective view of the user control unit 1100. The user control unit 1100 includes a left eye display 1112 and a right eye display 1114 for presenting the surgeon S with a coordinated stereoscopic view of the surgical site that enables depth perception. The user control unit 1100 further includes one or more input control devices 1116 (input device), which in turn cause the manipulator unit 1200 (shown in FIG. 1) to manipulate one or more tools. The input devices 1116 provide at least the same degrees of freedom as instruments 1400 with which they are associated to provide the surgeon S with telepresence, or the perception that the input devices 1116 are integral with (or are directly connected to) the instruments 1400. In this manner, the user control unit 1100 provides the surgeon S with a strong sense of directly controlling the instruments 1400. To this end, position, force, strain, or tactile feedback sensors (not shown) or any combination of such sensations, from the instruments 1400 back to the surgeon's hand or hands through the one or more input devices 1116.

The user control unit 1100 is shown in FIG. 1 as being in the same room as the patient so that the surgeon S can directly monitor the procedure, be physically present if necessary, and speak to an assistant directly rather than over the telephone or other communication medium. In other embodiments, however, the user control unit 1100 and the surgeon S can be in a different room, a completely different building, or other location remote from the patient, allowing for remote surgical procedures.

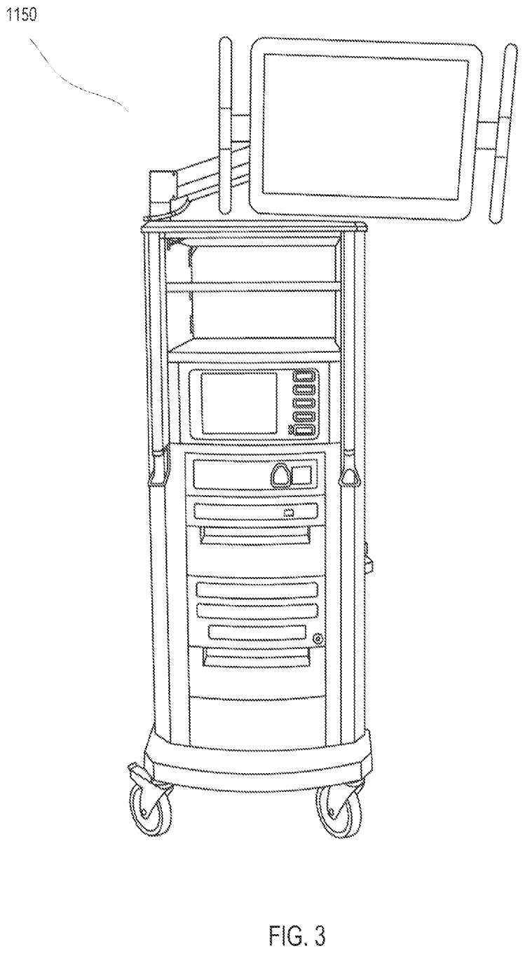

FIG. 3 is a perspective view of the auxiliary equipment unit 1150. The auxiliary equipment unit 1150 can be coupled with the endoscope (not shown) and can include one or more processors to process captured images for subsequent display, such as via the user control unit 1100, or on another suitable display located locally (e.g., on the unit 1150 itself as shown, on a wall-mounted display) and/or remotely. For example, where a stereoscopic endoscope is used, the auxiliary equipment unit 1150 can process the captured images to present the surgeon S with coordinated stereo images of the surgical site via the left eye display 1112 and the right eye display 1114. Such coordination can include alignment between the opposing images and can include adjusting the stereo working distance of the stereoscopic endoscope. As another example, image processing can include the use of previously determined camera calibration parameters to compensate for imaging errors of the image capture device, such as optical aberrations.

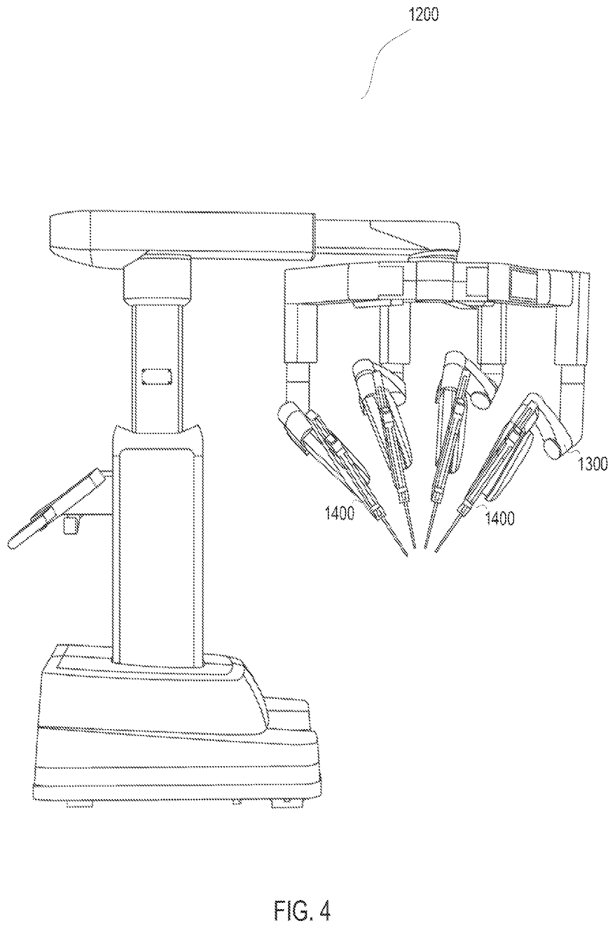

FIG. 4 shows a front perspective view of the manipulator unit 1200. The manipulator unit 1200 includes the components (e.g., arms, linkages, motors, sensors, and the like) to provide for the manipulation of the instruments 1400 and an imaging device (not shown), such as a stereoscopic endoscope, used for the capture of images of the site of the procedure. Specifically, the instruments 1400 and the imaging device can be manipulated by teleoperated mechanisms having one or more mechanical joints. Moreover, the instruments 1400 and the imaging device are positioned and manipulated through incisions or natural orifices in the patient P in a manner such that a center of motion remote from the manipulator and typically located at a position along the instrument shaft is maintained at the incision or orifice by either kinematic mechanical or software constraints. In this manner, the incision size can be minimized.

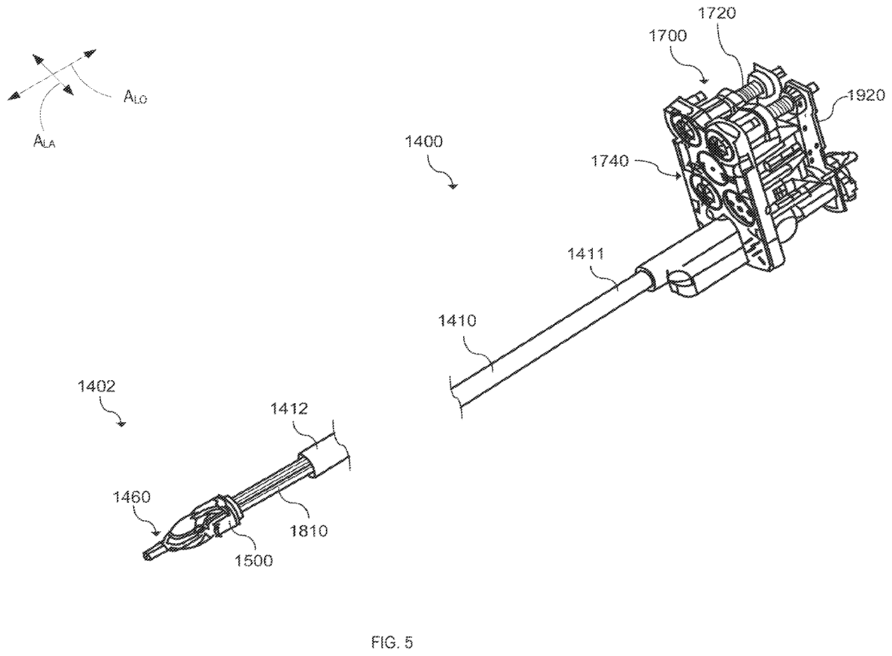

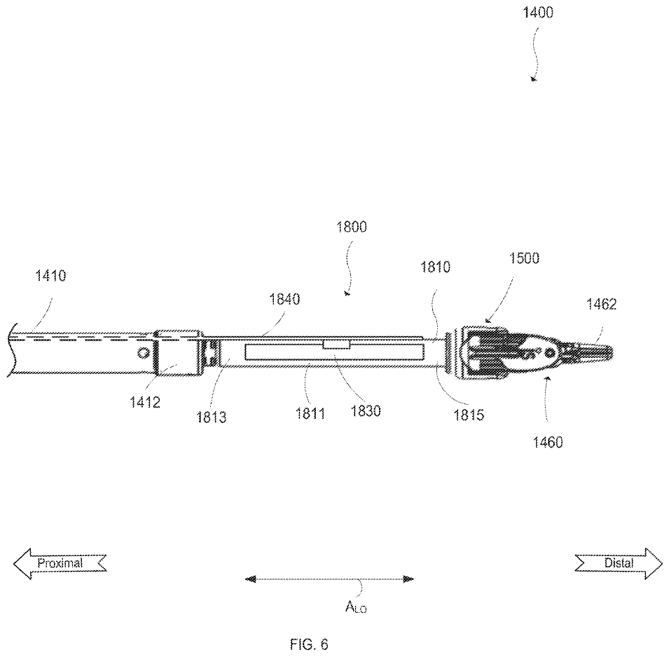

Referring now to FIGS. 5 and 6, a perspective view of the instrument 1400 is depicted in FIG. 5, and a side view of a portion of the instrument 1400 with an outer shaft portion removed is depicted in FIG. 6. In some embodiments, the instrument 1400 or any of the components therein are optionally parts of a surgical system that performs surgical procedures, and which can include a manipulator unit, a series of kinematic linkages, a set of cannulas, or the like. The instrument 1400 (and any of the instruments described herein) can be used in any suitable surgical system, such as the MIRS system 1000 shown and described above. As shown in FIG. 5, the instrument 1400 includes a proximal mechanical structure 1700 (depicted with an outer cover removed), a shaft 1410, a distal end portion 1402, and a set of cables (not shown). The cables function as tension elements that couple the proximal mechanical structure 1700 to the distal end portion 1402. In some embodiments, the distal end portion 1402 includes a distal wrist assembly 1500 and a distal end effector 1460. The instrument 1400 is configured such that movement of one or more of the cables produces movement of the end effector 1460 (e.g., pitch, yaw, or grip) about axes of a beam coordinate system BCS.

The proximal mechanical structure 1700 is configured to be removably coupled to the arm assembly 1300 manipulator unit 1200 (FIG. 4). The manipulator unit 1200 includes teleoperated actuators (e.g., motors with coupled drive discs) to provide controller motions to the instrument 1400, which translates into a variety of movements of a tool or tools at a distal end portion 1402 of the instrument 1400. When an instrument 1400 is coupled to the arm assembly 1300, input provided by a surgeon S to the user control unit 1100 (a “master” command) is translated into a corresponding action by the instrument 1400 (a “slave” response) via drive discs of the arm assembly 1300 that are operatively coupled instrument discs 1740 on the instrument 1400.

In some embodiments, the proximal mechanical structure 1700 includes a circuit board 1920 (e.g., a control board). The circuit board 1920 is communicatively coupled to a force sensor unit 1800 via a sensor cable 1840. The circuit board 1920 is configured to provide a voltage input to the strain sensor 1830 of the force sensor unit 1800 and to receive an output signal from the strain sensor 1830 that is indicative of a force affecting the distal end portion 1402 of the instrument 1400. Further details regarding the circuit board 1920 are provided in U.S. Provisional Patent Application No. 63/425,524 , filed Nov. 15, 2022, the disclosure of which is incorporated herein by reference for all purposes. Further details regarding the sensor cable are provided in U.S. Provisional Patent Application No. 63/425,520 , filed Nov. 15, 2022, the disclosure of which is incorporated herein by reference for all purposes.

Moreover, although the proximal mechanical structure 1700 is shown as including capstans 1720, in other embodiments, a mechanical structure can include one or more linear actuators that produce translation (linear motion) of a portion of the cables. Such proximal mechanical structures can include, for example, a gimbal, a lever, or any other suitable mechanism to directly pull (or release) an end portion of any of the cables. For example, in some embodiments, the proximal mechanical structure 1700 can include any of the proximal mechanical structures or components described in U.S. Patent Application Pub. No. US 2015/0047454 A1 (filed Aug. 15, 2014), entitled “Lever Actuated Gimbal Plate,” or U.S. Pat. No. 6,817,974 B2 (filed Jun. 28, 2001), entitled “Surgical Tool Having Positively Positionable Tendon-Actuated Multi-Disc Wrist Joint,” each of which is incorporated herein by reference in its entirety.

Referring still to FIGS. 5 and 6, the shaft 1410 can be any suitable elongated shaft that is coupled to the wrist assembly 1500 and to the proximal mechanical structure 1700. Specifically, the shaft 1410 includes a proximal end 1411 that is coupled to the proximal mechanical structure 1700, and a distal end portion 1412 that is coupled to the wrist assembly 1500 (e.g., a proximal link of the wrist assembly 1500). The shaft 1410 defines a passageway or series of passageways through which the cables and other components (e.g., the sensor cable 1840, electrical wires, ground wires, or the like) can be routed from the proximal mechanical structure 1700 to the wrist assembly 1500. In some embodiments, the shaft 1410 can be formed, at least in part with, for example, an electrically conductive material such as stainless steel. In such embodiments, the shaft may include any of an inner insulative cover or an outer insulative cover. Thus, the shaft 1410 can be a shaft assembly that includes multiple different components. For example, the shaft 1410 can include (or be coupled to) a spacer that provides the desired fluid seals, electrical isolation features, and any other desired components for coupling the wrist assembly 1500 to the shaft 1410. Similarly stated, although the wrist assembly 1500 (and other wrist assemblies or links described herein) are described as being coupled to the shaft 1410, it is understood that any of the wrist assemblies or links described herein can be coupled to the shaft via any suitable intermediate structure, such as a spacer and a cable guide, or the like.

As depicted in FIG. 6, the instrument 1400 (e.g., the force sensing medical instrument) includes a force sensor unit 1800. The force sensor unit includes a beam 1810, with one or more strain sensors 1830. The strain sensor 1830 can include a set of strain gauges (e.g., tension strain gauge resistor(s), compression strain gauge resistor(s), or both tension and compression strain gauge resistor(s)) arranged as at least one bridge circuit (e.g., a Wheatstone bridge) mounted on only a single surface along the beam 1810 or multiple surfaces along the beam 1810. In some embodiments, the end effector 1460 can be coupled at a distal end portion 1815 of the beam 1810 (e.g. at a distal end portion 1402 of the surgical instrument 1400) via the wrist assembly 1500. The shaft 1410 includes a distal end portion 1412 (e.g., an inner shaft) that is coupled to a proximal end portion 1813 of the beam 1810. In some embodiments, the distal end portion 1412 of the shaft 1410 is coupled to the proximal end portion 1813 of the beam 1810 via another coupling component (such as an anchor or coupler, not shown). In some embodiments, the force sensor unit 1800 can include any of the structures or components described in U.S. Patent Application Pub. No. US 2020/0278265 A1 (filed May. 13, 2020), entitled “Split Bridge Circuit Force Sensor,” which is incorporated herein by reference in its entirety.

In some embodiments, the end effector 1460 can include at least one tool member 1462 having a contact portion configured to engage or manipulate a target tissue during a surgical procedure. For example, in some embodiments, the contact portion can include an engagement surface that functions as a gripper, cutter, tissue manipulator, or the like. In other embodiments, the contact portion can be an energized tool member that is used for cauterization or electrosurgical procedures. The end effector 1460 may be operatively coupled to the proximal mechanical structure 1700 such that the tool member 1462 rotates relative to shaft 1410. In this manner, the contact portion of the tool member 1462 can be actuated to engage or manipulate a target tissue during a surgical procedure. The tool member 1462 (or any of the tool members described herein) can be any suitable medical tool member. Moreover, although only one tool member 1462 is identified, as shown, the instrument 1400 can include two tool members that cooperatively perform gripping or shearing functions. In other embodiments, an end effector can include more than two tool members.

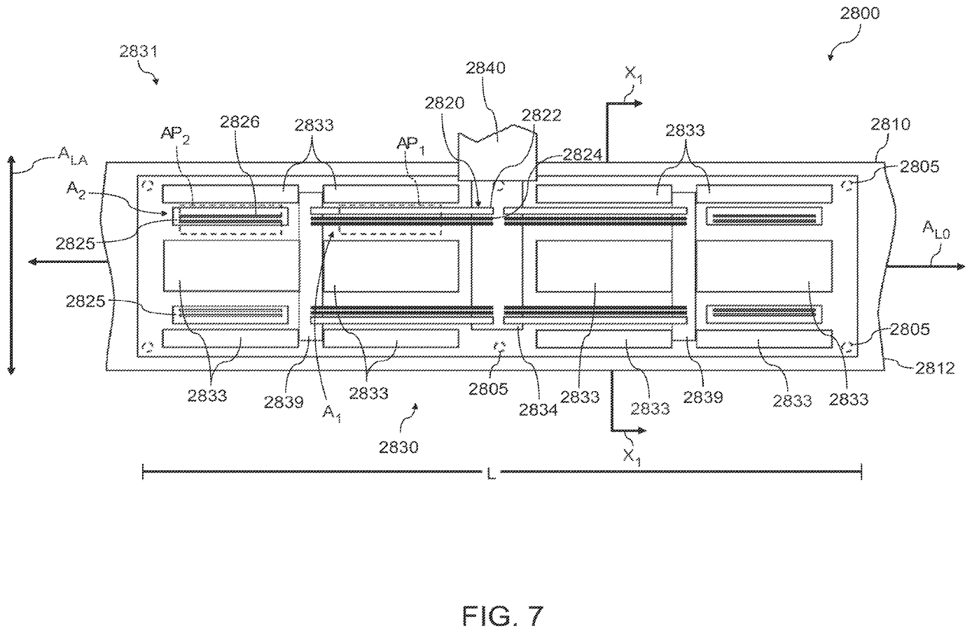

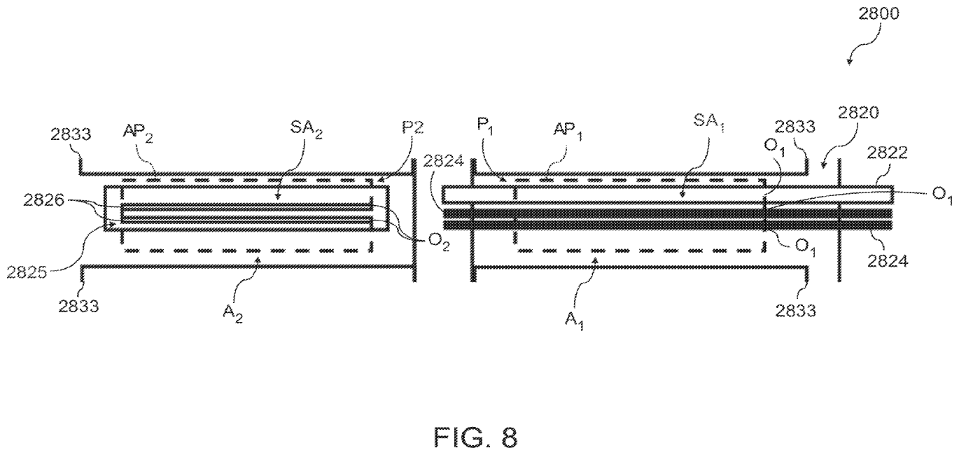

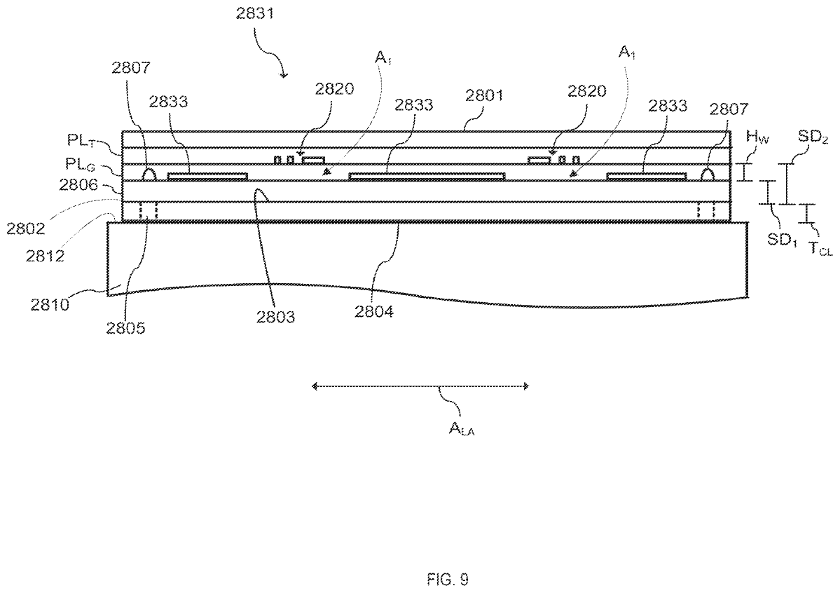

FIG. 7 is a schematic top view illustration and FIG. 9 is a schematic sectional view illustration of a force sensor unit 2800 for use with the instrument 1400 (or any of the instruments described herein) according to various embodiments. FIG. 8 is an enlarged view of a portion of the force sensor unit 2800. FIGS. 7 and 9 depict optional elements, components, and features of the various embodiments of the force sensor unit 2800. As such, it should be appreciated that some embodiments of the force sensor unit 2800 do not require each and every optional element, component, and/or feature depicted.

As depicted, the force sensor unit 2800 includes a beam 2810. The beam 2810 is a resiliently deflectable beam configured to bend or deflect in response to a load applied to a distal end portion of the instrument. A strain sensor 2830 is mounted on a lateral surface 2812 of the beam 2810 to sense strain that results from beam 2810 deflecting. The lateral surface 2812 extends along a longitudinal axis A_{LO} and a lateral axis A_{LA} of the beam 2810. The beam 2810 can, for example, couple the distal end portion of the instrument (e.g., distal end portion 1402 (FIG. 5)) to the shaft of the instrument (e.g., shaft 1410 (FIG. 5)) in a cantilevered configuration anchored at the proximal end portion of the beam 2810.

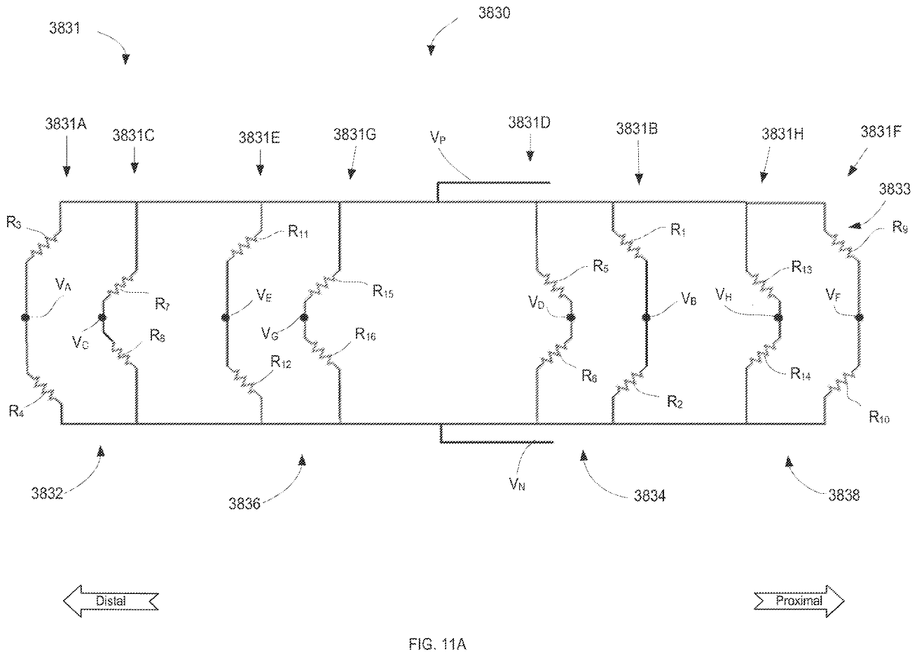

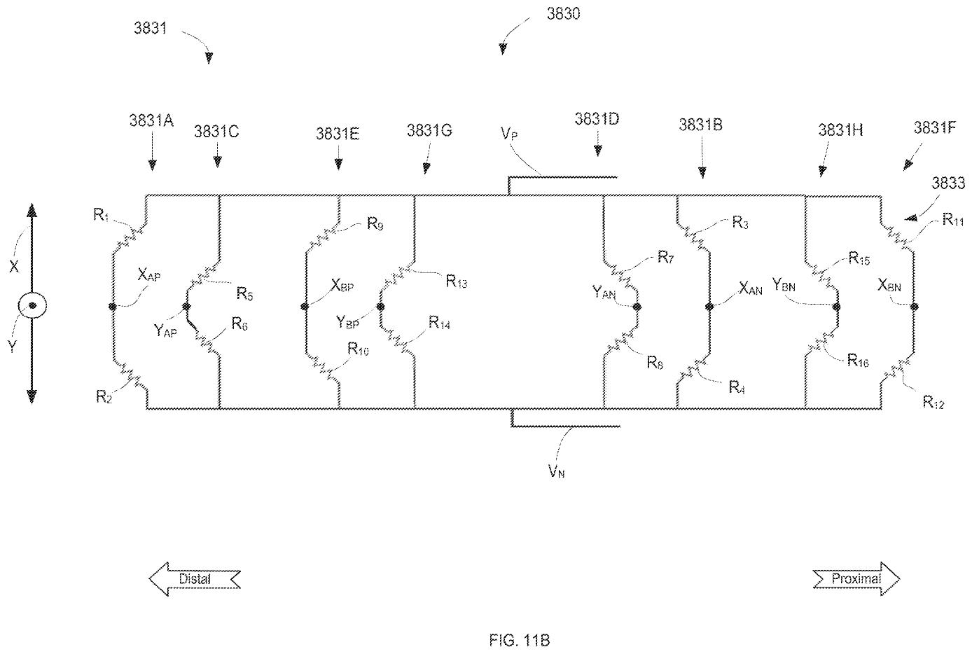

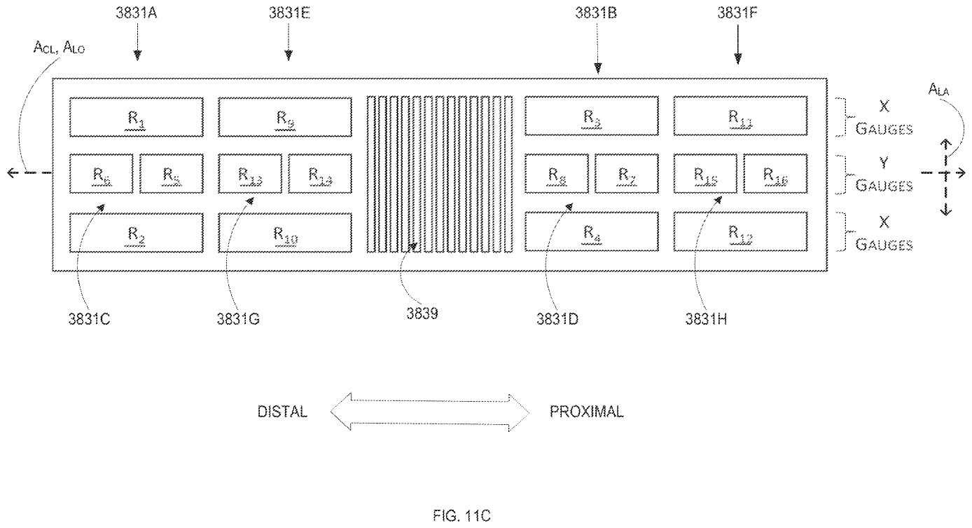

The strain sensor 2830 is optionally made of one or more electrical strain sensing circuits (e.g., half-bridge circuits 2831 (see e.g., FIG. 11)), and other strain sensor configurations are contemplated (e.g., piezoelectric sensors, and the like). As described herein, each half-bridge circuit 2831 (and also each strain sensor) includes one or more strain gauges 2833 (e.g., tension strain gauge resistor(s), compression strain gauge resistor(s), or both tension and compression strain gauge resistor(s)). It should be appreciated that the beam 2810 can include any number of strain sensors 2830 in various arrangements on one or more surfaces of the beam 2810. In some embodiments, the beam 2810 includes a single strain sensor 2830 that includes multiple bridge circuits split into multiple half-bridge circuits 2831, with each half-bridge circuit 2831 having at least two strain gauges 2833.

During certain operations, the beam 2810 can be capacitively coupled to the strain sensor 2830 when exposed to an electrical field. The orthogonal distances between the lateral surface 2812 of the beam 2810 and each of the strain gauges 2833 of the strain sensor 2830 can affect a current induced in the strain gauges 2833. When the distance between each of the strain gauges 2833 and the lateral surface 2812 is uniform, the induced current in each of the strain gauges 2833 is substantially equal to the induced current in each of the other strain gauges 2833. This equalization of the induced currents results in the canceling out or reduction of the effects of the electromagnetic interference in the output of the strain sensor 2830. In other words, since the induced current in each of the strain gauges 2833 has substantially the same value, the voltage of the output signals may have a greater magnitude, but the increase in voltage magnitude does not affect the voltage differential, and thus the indications of strain. However, variations in the flatness of the lateral surface 2812 and/or the thickness of an adhesive that couples the strain gauges 2833 to the beam 2810, can result in a lack of uniformity in the distance between each of the strain gauges 2833 and the lateral surface 2812 and corresponding variations in the induced currents, which, in turn, manifest in the output signals of the strain sensor 2830 as electromagnetic interference. As such, the force sensor unit 2800 disclosed herein, in some embodiments, utilizes an electrically conductive layer 2802 and an electrically insulative layer 2806 to facilitate uniform capacitive coupling when the force sensor unit 2800 is exposed to an electrical field.

As depicted in FIG. 9, the force sensor unit 2800 includes the electrically conductive layer 2802 over the lateral surface 2812 of the beam 2810. In some embodiments, the electrically conductive layer 2802 is mechanically bonded to the lateral surface 2812 of the beam 2810 along the length L (FIG. 7) of the electrically conductive layer 2802. In addition to the mechanical bonding, the electrically conductive layer 2802 is also electrically coupled to the beam 2810. In such an embodiments, the mechanical bonding facilitates the accurate measurement of strain resulting from the deflection of the beam 2810, while the electrical coupling transfers an electrical current from the beam 2810 to the electrically conductive layer 2802. As a result, the current conducted by the electrically conductive layer 2802 can cause the induced currents in the strain sensor 2830 instead of the current in the beam 2810. As such, the magnitude of the induced current is dependent, at least in part, on the separation distance (e.g., separation distance SD₁ and/or separation distance SD₂) between the components of the strain sensor 2830 and the electrically conductive layer 2802 rather than the distance between the components and the lateral surface 2812. In other words, with regards to capacitive coupling, the electrically conductive layer 2802 replaces the lateral surface 2812 of the beam 2810 due to the electrical coupling therebetween.

In order to electrically couple the electrically conductive layer 2802 to the lateral surface 2812, in some embodiments, the electrically conductive layer 2802 includes two or more weld locations 2805. The electrically conductive layer 2802 is electrically coupled to the beam 2810 at each of the weld locations 2805, such as via a weld. In some embodiments, the weld locations 2805 are positioned to produce a spatially uniform electrical coupling of the electrically conductive layer 2802 to the beam 2810. For example, as depicted in FIG. 7, in some embodiments, the force sensor unit 2800 has six weld locations 2805 that are distributed around a perimeter of the electrically conductive layer 2802. However, in additional embodiments, the force sensor unit 2800 can include three, four, eight, or ten weld locations 2805 distributed about the electrically conductive layer 2802. The distribution of the weld locations 2805 can facilitate a substantially uniform resistance to the current from the beam 2810 along the longitudinal length of the electrically conductive layer 2802 and across the lateral width of the electrically conductive layer 2802.

In some embodiments, the electrically conductive layer 2802 is mechanically bonded to the lateral surface 2812 of the beam 2810 via an adhesive. For example, the electrically conductive layer 2802 can be mechanically bonded to the lateral surface 2812 via an epoxy resin, an ethyl-based cyanoacrylate glue, a methyl-based cyanoacrylate glue, a phenolic resin, or other suitable adhesive. In some embodiments, the adhesive is distributed laterally (e.g., parallel to the lateral axis A_{LA}) across the lateral surface 2812 of the beam 2810 and longitudinally (e.g., parallel to the longitudinal axis A_{LO}) along the lateral surface 2812 of the beam 2810. The adhesive can, for example, cover at least 90 percent (e.g., at least 95 percent) of a surface 2804 of the electrically conductive layer 2802 that faces the beam 2810.

In some embodiments, the electrically conductive layer 2802 can have a top surface 2803 facing the strain sensor 2830 that is within a specified flatness tolerance. The flatness tolerance defines a maximal separation distance between a plane passing through the highest point of the surface and a parallel plane passing through the lowest point of the surface. The specified flatness tolerance can, for example, be 0.1 micrometers or less. The flatness tolerance of the top surface 2803 of the electrically conductive layer 2802 facilitates the positioning of the strain sensor 2830 at a uniform separation distance SD₁ from the electrically conductive layer 2802. Similarly, in some embodiments, the top surface 2803 of the electrically conductive layer 2802 can have a surface roughness that is less than 0.1 micrometers to facilitate the uniform separation distance SD₁. In some embodiments, the electrically conductive layer 2802 can have a thickness T_{CL} that is within a specified thickness range. The specified thickness range can, for example, be greater than 45 micrometers and less than or equal to 55 micrometers. The electrically conductive layer 2802 being within thickness range can facilitate a uniformity of stiffness and a uniformity of electrical resistance within the electrically conductive layer 2802.

As depicted in FIG. 9, the electrically insulative layer 2806 is positioned over the electrically conductive layer 2802. The strain sensor 2830 is positioned over (e.g., formed on) a length L of the electrically insulative layer 2806. In other words, the strain sensor 2830 is physically separated from the electrically conductive layer 2802 by the electrically insulative layer 2806, which precludes the establishment of a physical electrical connection between the strain sensor 2830 and the electrically conductive layer 2802. In other words, the electrically insulative layer 2806 precludes a conductive electrical coupling between the strain sensor 2830 and the electrically conductive layer 2802 and, thus, the beam 2810. In some embodiments, the electrically insulative layer 2806 has a uniform thickness. The uniform thickness of the electrically insulative layer 2806 establishes the uniform separation distance SD₁ between the strain sensor 2830 and the electrically conductive layer 2802. For example, in some embodiments, an electrically insulative layer 2806 of the uniform thickness is positioned over an electrically conductive layer 2802 that has a specified flatness in order to position each strain gauge 2833 of the strain sensor 2830 at an equal distance (i.e., the uniform separation distance SD₁) from the electrically conductive layer 2802.

In some embodiments, the electrically conductive layer 2802 can, for example, be a stainless steel, such as grade 304 austenitic stainless steel, grade 316 austenitic stainless steel, or other suitable stainless steel alloy. In some embodiments, the electrically insulative layer 2806 can, for example, be a polyimide film or other suitable electrically insulative film.

Referring again to FIGS. 7-9, in some embodiments, the strain sensor 2830 includes a bridge circuit 2831, a set of electrical pads 2839 (e.g., contacts, tap points, or pickup points), and an electrical trace structure 2820. As depicted, the bridge circuit 2831 (e.g., a set of split, half-bridge circuits distributed along the longitudinal axis A_{LO} of the beam 2810) includes a set of strain gauges 2833 that are over (e.g., formed on) the electrically insulative layer 2806. The bridge circuit 2831 has a uniform separation distance SD₁ from the electrically conductive layer 2802. The electrical trace structure 2820 is electrically coupled between the sensor cable 2840 and the electrical pads 2839. As such, the electrical trace structure 2820 can provide an input voltage to the bridge circuit 2831 and can transmit an output signal indicative of strain to the sensor cable 2840.

In some embodiments, the strain gauges 2833 are positioned on a gauge plane PL_{G} that is parallel to the electrically conductive layer 2802. Being parallel with the electrically conductive layer 2802 the gauge plane PL_{G} is separated from the electrically conductive layer 2802 by the uniform separation distance SD₁. The electrical trace structure 2820 is similarly positioned on a trace plane PL_{T}. The trace plane PL_{T} is parallel to the gauge plane PL_{G}. The trace plane PL_{T} is separated from the electrically conductive layer 2802 by a uniform separation distance SD₂. In some embodiments, the distance between the trace plane PL_{T} and the electrically conductive layer 2802 is greater than the distance between the gauge plane PL_{G} and the electrically conductive layer 2802. As depicted, the electrical trace structure 2820 is laterally offset from each strain gauge 2833. In some embodiments, portions of the electrical trace structure 2820 can be disposed over portions of the strain gauges 2822 while maintaining a lateral offset. This positioning of the electrical trace structure 2820 facilitates a uniformity in a stiffness of the strain sensor 2830 that extends parallel to the lateral surface 2812 of the beam 2810. The offset distance between the electrical trace structures and the strain gauges also mitigates potential interference between the traces and the strain gauges that could result from mismatched thermal expansion effects. The uniformity of the stiffness of the strain sensor 2830 facilitates the accurate measurement of the strain developed in the beam 2810 in response to a load applied to the instrument. In contrast, localized stiffness concentrations resulting from the stacking or overlapping of components, such as the positioning of the electrical trace structure 2820 at the same lateral and longitudinal point as a strain gauges 2833 can establish a localized stiffness concentration. The localized stiffness concentration can affect the response of the co-located strain gauge 2833 to the deflection of the beam 2810 and, thus, the magnitude of the strain indicated by the strain gauge 2833.

In some embodiments, the force sensor unit 2800 includes an enclosure layer 2801. The enclosure layer 2801 covers the strain sensor 2830. The enclosure layer 2801 can be a flexible protective covering that seals the strain sensor 2830 to the beam 2810 to preclude the introduction of liquids to the strain sensor 2830. The enclosure layer 2801 can, in some embodiments, have a uniform thickness. The uniform thickness of the enclosure layer 2801 can facilitate a uniformity in the stiffness of the strain sensor 2830.

As depicted in FIG. 9, in some embodiments, the bridge circuit 2831 can be surrounded by a wall 2807. In other words, the wall 2807 can form a perimeter around portions of the strain sensor 2830. The wall 2807 can be positioned on the gauge plane PL_{G} and extend away from the lateral surface 2812. The wall 2807 can have a height H_{W} that is equal to or greater than a thickness of the strain gauges 2833. The wall 2807 can, for example, be formed from a copper-nickel alloy wire. The presence of the wall 2807 can form a barrier to liquid intrusion, thereby increasing a durability of the strain sensor 2830. For example, the wall 2807 can shield the strain gauges from direct contact with high-temperature cleaning fluids (e.g., steam under pressure) during post-procedure processing (e.g., autoclaving).

Referring again to FIGS. 7 and 8, in order to mitigate the potential electromagnetic interference stemming from inductive coupling, in some embodiments, the force sensor unit 2800 includes the strain sensor 2830 mechanically coupled to the beam 2810. The strain sensor includes a first region A₁ and a second region A₂. The strain sensor 2830 also includes the bridge circuit 2831, the electrical trace structure 2820 and a balancing structure 2825. The electrical trace structure 2820 is positioned over the first region A₁ and is electrically coupled to the bridge circuit 2831. The balancing structure 2825 is positioned over the second region A₂ and has an absence of physical electrical connections with any other component of the force sensor unit 2800. In other words, the balancing structure 2825 can be a conductive component (e.g., electrical trace(s)) that does not form a conductive path with any other conductive component of the force sensor unit 2800. In some embodiments, the balancing structure 2825 can be a conductive copper trace that is completely surrounded by an insulating material. For example, the balancing structure 2825 can be formed as a trace structure that is not in physical contact with any other electrically conductive component of the force sensor unit 2800. In some embodiments, the balancing structure 2825 can be a conductive electrical trace(s) connected (e.g., by vias) to the electrically conductive layer 2802 (e.g., to be grounded).

As depicted in FIG. 8, in some embodiments, the electrical trace structure 2820 includes a first area portion AP₁. Similarly, the balancing structure 2825 includes a second area portion AP₂. An outline O₁ of the first area portion AP₁ of the electrical trace structure 2820 defines a first pattern P₁. The first pattern P₁ has a first surface area SA₁. The outline O₁ of the first area portion AP₁ can, for example, be defined by each edge (e.g., perimeter edges) of the portions of electrically conductive traces (e.g., deposited copper traces) positioned within the first area portion AP₁. Accordingly, the first surface area SA₁ can include the combined surface area of the portions of the electrically conductive traces within the first area portion AP₁. An outline O₂ of the second area portion AP₂ of the balancing structure 2825 defines a second pattern P₂. The second pattern P₂ as a second surface area SA₂. The outline O₂ of the second area portion AP₂ can, for example, be defined by a combination of the edges of the conductive material (e.g., copper) that are in contact with an insulative material of the trace layer PL_{T} within the second area portion AP₂. Accordingly, the second surface area SA₂ can include the surface area of the conductive material that is within the second area portion AP₂.

As depicted in FIG. 8, in some embodiments, the second pattern P₂ of the balancing structure 2825 matches the first pattern P₁ of the electrical trace structure 2820. Similarly, the second surface area SA₂ of the balancing structure 2825 is substantially equal to the first surface area SA₁ of the electrical trace structure 2820. The second pattern P₂ of the balancing structure 2825 can, for example, be configured to generate a first voltage change in the strain sensor 2830 that is proportional to a second voltage change generated in the strain sensor 2830 by the electrical trace structure 2820. In other words, the second pattern P₂ can be configured such that the balancing structure 2825 has substantially the same inductive coupling with a first adjacent component that the electrical trace structure 2820 has with a second adjacent component. In some embodiments, a longitudinal axis of the second pattern P₂ of the balancing structure 2825 is aligned with a longitudinal axis of the first pattern P₁ of the electrical trace structure 2820. In other words, the second pattern P₂ and the first pattern P₁ can be located at the same lateral position and can extend parallel to strain gauges 2833 of the strain sensor 2830. It should be appreciated that the establishment of symmetry between the first pattern P₁ and the second pattern P₂ and the first surface area SA₁ and the second surface area SA₂ facilitates the mitigation of the electromagnetic interference by providing substantially equal induced currents to the positive and negative traces of the strain sensor 2830. In addition, this structural symmetry around each strain gauge operates in the thermal space as well, with each gauge seeing the same environment of temperature induced strain.

In other embodiments, the first and second patterns can be located at different lateral positions with respects to the strain sensor 2830 and extend parallel and/or transverse to the strain gauges 2833. In other words, the longitudinal axes of the first and second patterns are spaced apart laterally from each other. In yet other embodiments, the first pattern can be disposed and spaced apart between the second pattern and a third pattern of a second balancing structure (not shown). For example, the second and third patterns of the balancing structures can be located on opposite lateral sides of and spaced apart from the first pattern of the electrical trace structure. The first pattern of the electrical trace structure can extend along a longitudinal midline of the strain sensor. The second and third patterns can extend along parallel, longitudinal axes spaced apart from the longitudinal midline of the strain sensor. As such, the one or more electrically conductive traces of the first, second, and/or third patterns are spaced apart from each other laterally. The first, second, and/or third patterns as described herein can extend in a proximal to distal direction across a substantial length of the strain sensor. The second and third patterns of the balancing structures can have a combined, total surface area (e.g., area of the electrically conductive trace(s) of the balancing structures) substantially equal to the surface area of the first pattern (e.g., area of the electrically conductive trace(s) of the electrical trace structure). The first, second, and/or third patterns as described herein can include one or more electrically conductive traces.

In some embodiments, the electrical trace structure 2820 includes an input trace 2822. The electrical trace structure 2820 can also include one or more measurement traces 2824 (e.g., signal traces). The input trace 2822 is configured to deliver an input voltage (e.g., an excitation voltage) from the sensor cable 2840 to one or more half-bridge circuits 2831. The measurement trace 2824 is configured to deliver an output signal from the split half-bridge circuit 2831 to the sensor cable 2840. As depicted, the input trace 2822 can have a lateral width that is greater than a lateral width of the measurement trace 2824 when oriented parallel to the longitudinal axis A_{LO}. The first area portion AP₁ of the electrical trace structure 2820 includes a portion of the input trace 2822 and a portion of the measurement trace 2824. In some embodiments, the second pattern P₂ of the balancing structure 2825 defines a void 2826 that corresponds to the separation between the input trace 2822 and the measurement trace 2824.

As depicted in FIGS. 7 and 9, the strain sensor 2830 can include multiple half-bridge circuits 2831. As is described more fully below, the bridge circuits 2831 can for example, be arranged as a four bridge-circuit configuration split into eight half-bridge circuits that each include a set of strain gauges 2833. In such embodiments, the strain sensor 2830 can include four electrical trace structures 2820 and four balancing structures 2825. Each of the electrical trace structures 2820 and the balancing structures 2825 can be positioned on the trace plane PL_{T} (e.g., within a single trace layer) that is over and parallel to the gauge plane PL_{G}. Each of the electrical trace structures 2820 and the balancing structures 2825 is laterally offset from each strain gauge 2833. This lateral offset facilitates a uniformity in the stiffness (e.g., rigidity) of the strain sensor. In other words, offsetting the electrical trace structures 2820 and the balancing structures 2825 from the strain gauges 2833 precludes the generation of stiffness concentrations at the points of overlap between the strain gauges 2833 and the electrical trace structures 2820 and/or the balancing structures 2825.

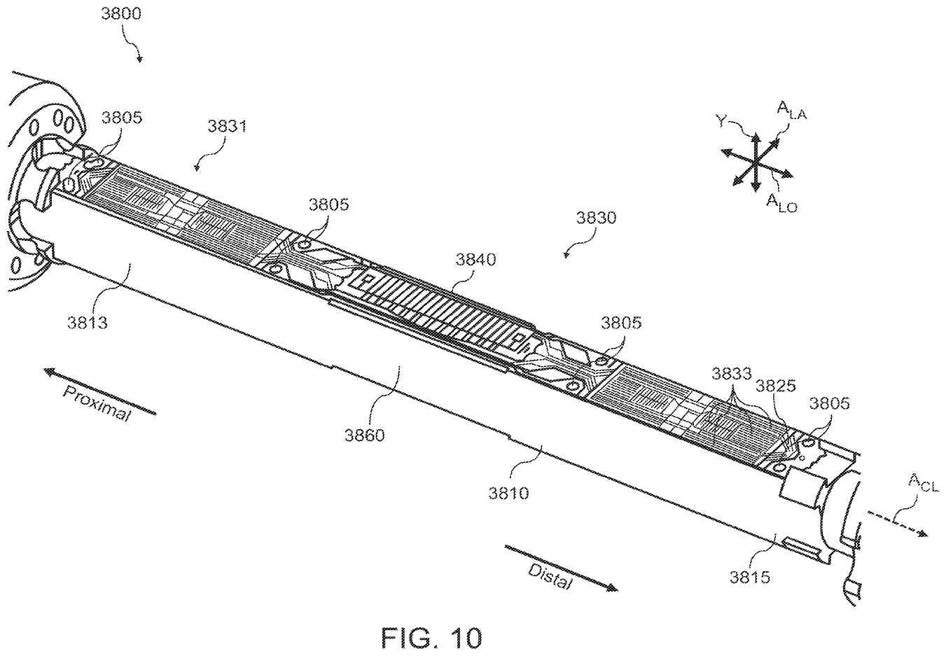

FIGS. 10-14 depict various view of aspects of a force sensor unit 3800 for use with a force sensing medical instrument, such as instrument 1400 described herein. In some embodiments, the force sensor unit 3800 or any of the components therein are optionally parts of a surgical system that performs surgical procedures. The surgical system may include a manipulator unit, a series of kinematic linkages, a series of cannulas, or the like. The force sensor unit 3800 (and any of the force sensor units described herein) can be used in any suitable surgical system, such as the MIRS system 1000 shown and described above to mitigate the effects of electromagnetic interference when the instrument is exposed to an electrical field.

FIG. 10 is a perspective view of the force sensor unit 3800 according to an embodiment. The force sensor unit 3800 includes a beam 3810, with one or more strain sensors 3830. The strain sensor 3830 can include a set of strain gauges 3833 (e.g., tension strain gauge resistor(s), compression strain gauge resistor(s), or both tension and compression strain gauge resistor(s)) arranged as at least one bridge circuit 3831 (e.g., a Wheatstone bridge) mounted on a surface along the beam 3810. For example, the strain sensor 3830 can have a longitudinal axis that is arranged parallel to a longitudinal axis of the beam 3810. While illustrated as mounted on only one surface along the beam 3810, in other embodiments, the strain gauges 3833 can be mounted on one, two, or more surfaces along the beam 3810. In some embodiments, an end effector (e.g., end effector 1460) can be coupled at a distal end portion 3815 of the beam 3810 (e.g. at a distal end portion 1402 of the surgical instrument 1400) via a wrist assembly (e.g., wrist assembly 1500). In some embodiments, a distal end portion of an instrument shaft (e.g., shaft 1410) is coupled to the proximal end portion 3813 of the beam 3810 via a coupling component (such as an anchor or coupler, not shown). In some embodiments, the force sensor unit 3800 can include any of the structures or components described in U.S. Patent Application Pub. No. US 2020/0278265 A1 (filed May. 13, 2020), entitled “Split Bridge Circuit Force Sensor,” which is incorporated herein by reference in its entirety.

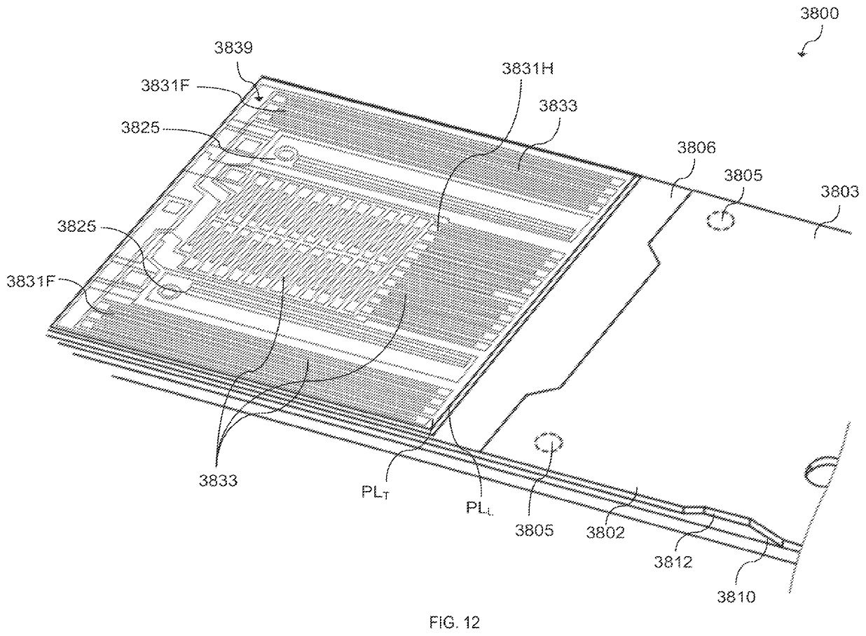

The beam 3810 is a resiliently deflectable beam configured to bend or deflect in response to a load applied to a distal end portion of the instrument. A strain sensor 3830 is mounted on a lateral surface 3812 (FIG. 12) of the beam 3810 to sense strain that results from beam 3810 deflecting. The lateral surface 3812 extends along a longitudinal axis A_{LO} and a lateral axis A_{LA} of the beam 3810. The beam 3810 can, for example, couple the distal end portion of the instrument (e.g., distal end portion 1402 (FIG. 5)) to the shaft of the instrument (e.g., shaft 1410 (FIG. 5)) in a cantilevered configuration anchored at the proximal end portion 3813 of the beam 3810.

The strain sensor 3830 is optionally made of one or more electrical strain sensing circuits (e.g., four full-bridge circuits formed from eight half-bridge circuits 3831 (FIG. 11)), and other strain sensor configurations are contemplated (e.g., piezoelectric sensors, and the like). As described herein, each bridge circuit 3831 (and also each strain sensor) includes one or more strain gauges 3833 (e.g., tension strain gauge resistor(s), compression strain gauge resistor(s), or both tension and compression strain gauge resistor(s)). It should be appreciated that the beam 3810 can include any number of strain sensors 3830 in various arrangements. In some embodiments, the beam 3810 includes a single strain sensor 3830 that includes multiple split half-bridge circuits 3831, with each split, half-bridge circuit 3831 having at least two strain gauges 3833.

FIGS. 11A and 11B are diagrammatic illustrations of configurations of the strain sensor 3830 depicted in FIG. 10, showing eight half-bridge circuits 3831A-3831H having a set of strain gauges 3833 (R₁-R₁₆) of a four full bridge circuit configuration. The eight half-bridge circuits 3831 include a first half-bridge circuit 3831A, a second half-bridge circuit 3831B, a third half-bridge circuit 3831C, a fourth half-bridge circuit 3831D, a fifth half-bridge circuit 3831E, a sixth half-bridge circuit 3831F, a seventh half-bridge circuit 3831G, and an eighth half-bridge circuit 3831H. In order to detect strain, an input voltage (eg, positive input voltage V_{P} and negative input voltage V_{N}) is provided to the eight half-bridge circuits 3831(A-H), and an output voltage (e.g., V_{A}, V_{B}, V_{C}, V_{D}, V_{E}, V_{F}, V_{G}, and V_{H} (V_{A-H})) can then be measured for each of the eight half-bridge circuits 3831(A-H). Various combinations of the output voltages (V_{A-H}) may be employed by the controller to determine a magnitude of the force affecting the instrument based on the sensed strain.

As depicted in FIG. 11A, in some embodiments, the first half-bridge circuit 3831A and the third half-bridge circuit 3831C are arranged as a primary distal bridge-circuit combination 3832, while the second half-bridge circuit 3831B and the fourth half-bridge circuit 3831D are arranged as a primary proximal bridge-circuit combination 3834. Additionally, in some embodiments, the fifth half-bridge circuit 3831E and the seventh half-bridge circuit 3831G are arranged as a secondary distal bridge-circuit combination 3836, while the sixth half-bridge circuit 3831F and the eighth half-bridge circuit 3831H are arranged as the secondary proximal bridge-circuit combination 3838. An output of the secondary distal bridge-circuit combination 3836 is redundant to a corresponding output of the primary distal bridge-circuit combination 3832. Similarly, an output of the secondary proximal bridge-circuit combination 3838 is redundant to a corresponding output of the primary proximal bridge-circuit combination 3834. In other words, absent a sensor malfunction, the outputs of the secondary distal bridge-circuit combination 3836 and the secondary proximal bridge-circuit combination 3838 equal the outputs of the primary distal bridge-circuit combination 3832 and the primary proximal bridge-circuit combination 3834.

As depicted in FIG. 11A, the first half-bridge circuit 3831A can include the third strain gauge resistor (R₃) and the fourth strain gauge resistor (R₄). The third and fourth strain gauge resistors (R₃, R₄) can be positioned on opposite sides of a beam center axis A_{CL} (FIG. 10) (e.g., a longitudinal axis A_{LO} that is centered laterally on a lateral surface 3812 (FIG. 12) of the beam 3810) and equidistant from the center axis. For example, the third and fourth strain gauge resistors (R₃, R₄) can be positioned equidistant between the beam center axis A_{CL} and a side edge of the surface to which they are mounted. In some embodiments, the third and fourth strain gauge resistors (R₃, R₄) can be positioned at the same proximal position along the beam center axis A_{CL}. In some embodiments, the third and fourth strain gauge resistors (R₃, R₄) are both the same type of strain gauge resistor (e.g., are both tension strain gauge resistors).

As further depicted in FIG. 11A, the third half-bridge circuit 3831C can include the seventh strain gauge resistor (R₇) and the eighth strain gauge resistor (R₈). The seventh and eighth strain gauge resistors (R₇, R₈) are positioned in axial alignment with the beam center axis A_{CL}. In some embodiments, a portion of the eighth strain gauge resistor (R₈) is positioned axially between the portions of the seventh strain gauge resistor (R₇), and a portion of the seventh strain gauge resistor (R₇) is positioned axially between the portions of the eighth strain gauge resistor (R₈). In some embodiments, one of the seventh and eighth strain gauge resistors (R₇, R₈) is a tension strain gauge resistor while the other is a compression strain gauge resistor.

As depicted in FIG. 11A, the second half-bridge circuit 3831B can include the first strain gauge resistor (R₁) and the second strain gauge resistor (R₂). The first and second strain gauge resistors (R₁, R₂) can be positioned on opposite sides of the beam center axis A_{CL} and equidistant from the center axis. For example, the first and second strain gauge resistors (R₁, R₂) can be positioned equidistant between the beam center axis A_{CL} and a side edge of the surface to which they are mounted. In some embodiments, the first and second strain gauge resistors (R₁, R₂) can be positioned at the same proximal position along the beam center axis A_{CL}. In some embodiments, the first and second strain gauge resistors (R₁, R₂) are both the same type of strain gauge resistor (e.g., are both tension strain gauge resistors).

As further depicted in FIG. 11A, the fourth half-bridge circuit 3831D can include the fifth strain gauge resistor (R₅) and the sixth strain gauge resistor (R₆). The fifth and sixth strain gauge resistors (R₅, R₆) are positioned in axial alignment with the beam center axis A_{CL}. In some embodiments, a portion of the sixth strain gauge resistor (R₆) is positioned axially between the portions of the fifth strain gauge resistor (R₅), and a portion of the fifth strain gauge resistor (R₅) is positioned axially between the portions of the sixth strain gauge resistor (R₆). One of the fifth and sixth strain gauge resistors (R₅, R₆) is a tension strain gauge resistor while the other is a compression strain gauge resistor.

Referring again to FIG. 11A, as depicted, the fifth half-bridge circuit 3831E can include the eleventh strain gauge resistor (R₁₁) and the twelfth strain gauge resistor (R₁₂). The eleventh and twelfth strain gauge resistors (R₁₁, R₁₂) can be positioned on opposite sides of the beam center axis A_{CL} and equidistant from the center axis. For example, the eleventh and twelfth strain gauge resistors (R₁₁, R₁₂) can be positioned equidistant between the beam center axis A_{CL} and a side edge of the surface to which they are mounted. In some embodiments, the eleventh and twelfth strain gauge resistors (R₁₁, R₁₂) can be positioned at the same proximal position along the beam center axis A_{CL}. In some embodiments, the eleventh and twelfth strain gauge resistors (R₁₁, R₁₂) are both the same type of strain gauge resistor (e.g., are both tension strain gauge resistors). The fifth half-bridge circuit 3831E is positioned distally relative to the first half-bridge circuit 3831A.

As further depicted in FIG. 11A, the seventh half-bridge circuit 3831G can include the fifteenth strain gauge resistor (R₁₅) and the sixteenth strain gauge resistor (R₁₆). The fifteenth and sixteenth strain gauge resistors (R₁₅, R₁₆) are positioned in axial alignment with the beam center axis A_{CL}. In some embodiments, a portion of the fifteenth strain gauge resistor (R₁₅) is positioned axially between the portions of the sixteenth strain gauge resistor (R₁₆), and a portion of the sixteenth strain gauge resistor (R₁₆) is positioned axially between the portions of the fifteenth strain gauge resistor (R₁₅). One of the fifteenth and sixteenth strain gauge resistors (R₁₅, R₁₆) is a tension strain gauge resistor while the other is a compression strain gauge resistor. The seventh half-bridge circuit 3831G is positioned distally relative to the third half-bridge circuit 3831C.

As depicted in FIG. 11A, the sixth half-bridge circuit 3831F can include the 9^{th} strain gauge resistor (R₉) and the tenth strain gauge resistor (R₁₀). The ninth and tenth strain gauge resistors (R₉, R₁₀) can be positioned on opposite sides of the beam center axis A_{CL} and equidistant from the center axis. For example, the ninth and tenth strain gauge resistors (R₉, R₁₀) can be positioned equidistant between the beam center axis A_{CL} and a side edge of the surface to which they are mounted. In some embodiments, the ninth and tenth strain gauge resistors (R₉, R₁₀) can be positioned at the same proximal position along the beam center axis A_{CL}. In some embodiments, the ninth and tenth strain gauge resistors (R₉, R₁₀) are both the same type of strain gauge resistor (e.g., are both tension strain gauge resistors). The sixth half-bridge circuit 3831F is positioned distally relative to the second half-bridge circuit 3831B.

As further depicted in FIG. 11A, the eighth half-bridge circuit 3831H can include the thirteenth strain gauge resistor (R₁₃) and the fourteenth strain gauge resistor (R₁₄). The thirteenth and fourteenth strain gauge resistors (R₁₃, R₁₄) are positioned in axial alignment with the beam center axis A_{CL}. In some embodiments, a portion of the thirteenth strain gauge resistor (R₁₃) is positioned axially between the portions of the fourteenth strain gauge resistor (R₁₄), and a portion of the fourteenth strain gauge resistor (R₁₄) is positioned axially between the portions of the thirteenth strain gauge resistor (R₁₃). One of the thirteenth and fourteenth strain gauge resistors (R₁₃, R₁₄) is a tension strain gauge resistor while the other is a compression strain gauge resistor. The eighth half-bridge circuit 3831H is positioned distally relative to the fourth half-bridge circuit 3831D.

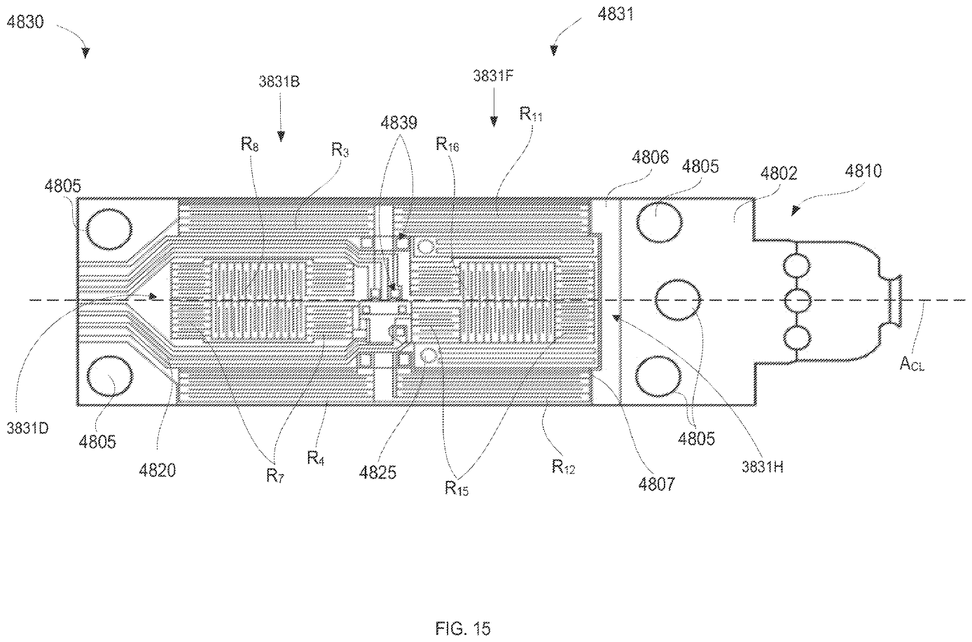

As depicted in FIGS. 11B and 11C, in some embodiments, the strain sensor includes a four full-bridge circuit arrangement with each full bridge circuit including two half-bridge circuits for a total of eight half-bridge circuits. The corresponding half-bridge circuits of each full bridge circuit are located on opposite end portions of the strain sensor (e.g., on a distal end portion and a proximal end portion) in contrast to the arrangement depicted in FIG. 11A. The first half-bridge circuit 3831A is positioned at the distal end portion 3815 of the beam 3810, while the second half-bridge circuit 3831B is positioned at the proximal end portion 3813 of the beam 3810. The sensor cable 2840, pads 3839, and/or anisotropic conductive film (ACF) can extend or be disposed therebetween (e.g., separating the first and second-half-bridges, the distal and proximal end portion half-bridges) as described in more detail below. The first half-bridge circuit 3831A and the second half-bridge circuit 3831B can be electrically coupled to form a first primary-full-bridge circuit. The first primary-full-bridge circuit can be configured to measure strain imparted along a first axis. The first axis can, for example, be lateral to the lateral face 3812 of the beam 3810 (e.g., in the direction of the lateral axis A_{LA}). In some embodiments, the first axis is an X-axis and the strain gauges of the first primary-full-bridge circuit can each be tension strain gauge resistors (e.g., to measure strain along the X-axis). In some embodiments, the tension strain gauge resistors described herein can have elongated portions aligned in parallel and coupled end-to-end to form a serpentine or snake-like configuration. The elongated portions of the tension gauge resistors can extend or be aligned parallel to the longitudinal axis A_{LO}. (See e.g., strain gauge resistors R₃, R₄, R₇, R₁₁, R₁₂, and R₁₅ as depicted in FIG. 15).

As depicted, first half-bridge circuit 3831A can include the first strain gauge resistor (R₁) and the second strain gauge resistor (R₂). The first and second strain gauge resistors (R₁, R₂) can be positioned on opposite sides of the beam center axis A_{CL} and equidistant from the center axis. For example, the first and second strain gauge resistors (R₁, R₂) can be positioned equidistant between the beam center axis A_{CL} and a side edge of the surface to which they are mounted. In some embodiments, the first and second strain gauge resistors (R₁, R₂) can be positioned at the same distal position along the beam center axis A_{CL}. In some embodiments, the first and second strain gauge resistors (R₁, R₂) are both the same type of strain gauge resistor (e.g., are both tension strain gauge resistors).

As further depicted, the second half-bridge circuit 3831B can include the third strain gauge resistor (R₃) and the fourth strain gauge resistor (R₄). The third and fourth strain gauge resistors (R₃, R₄) can be positioned on opposite sides of a beam center axis A_{CL} (e.g., a longitudinal axis A_{LO} that is centered laterally on a lateral surface 3812 (FIG. 12) of the beam 3810) and equidistant from the center axis. For example, the third and fourth strain gauge resistors (R₃, R₄) can be positioned equidistant between the beam center axis A_{CL} and a side edge of the surface to which they are mounted. In some embodiments, the third and fourth strain gauge resistors (R₃, R₄) can be positioned at the same proximal position along the beam center axis A_{CL}. In some embodiments, the third and fourth strain gauge resistors (R₃, R₄) are both the same type of strain gauge resistor (e.g., are both tension strain gauge resistors).

As depicted in FIGS. 11B and 11C, in some embodiments, the third half-bridge circuit 3831C is positioned at the distal end portion 3815 of the beam 3810, while the fourth half-bridge circuit 3831D is positioned at the proximal end portion 3813 of the beam 3810. The third half-bridge circuit 3831C and the fourth half-bridge circuit 3831D can be electrically coupled to form a second primary-full-bridge circuit. The second primary-full-bridge circuit can be configured to measure strain imparted along a second axis orthogonal to the first axis (e.g., to measure strain along the second axis). The second axis can, for example, be normal to the lateral face 3812 of the beam 3810. In some embodiments, the second axis is a Y-axis and the strain gauges of the second primary-full-bridge circuit can be a combination of tension strain gauge resistors and compression strain gauge resistors. As discussed above with respect to the first primary full-bridge circuit, the tension strain gauge resistors described herein can have elongated portions aligned in parallel and coupled end-to-end to form a serpentine or snake-like configuration. The elongated portions of the tension gauge resistors can extend or be aligned parallel to the longitudinal axis A_{LO}. Similarly, the compression strain gauge resistors described herein can also have elongated portions aligned in parallel and coupled end-to-end to form a serpentine or snake-like configuration. However, the elongated portions of the compression gauge resistors described herein can extend or be aligned transverse to the longitudinal axis A_{LO} (e.g., parallel to the lateral axis A_{LA}). (See e.g., strain gauge resistors R₈ and R₁₆ as depicted in FIG. 15)

As depicted, the third half-bridge circuit 3831C can include the fifth strain gauge resistor (R₅) and the sixth strain gauge resistor (R₆). The fifth and sixth strain gauge resistors (R₅, R₆) are positioned in axial alignment with the beam center axis A_{CL}. In some embodiments, a portion of the sixth strain gauge resistor (R₆) is positioned axially between the portions of the fifth strain gauge resistor (R₅), and/or a portion of the fifth strain gauge resistor (R₅) is positioned axially between the portions of the sixth strain gauge resistor (R₆). In some embodiments, one of the fifth and sixth strain gauge resistors (R₅, R₆) is a tension strain gauge resistor while the other is a compression strain gauge resistor.

As further depicted, the fourth half-bridge circuit 3831D can include the seventh strain gauge resistor (R₇) and the eighth strain gauge resistor (R₈). The seventh and eighth strain gauge resistors (R₇, R₈) are positioned in axial alignment with the beam center axis A_{CL}. In some embodiments, a portion of the eighth strain gauge resistor (R₈) is positioned axially between the portions of the seventh strain gauge resistor (R₇) (e.g., as illustrated in FIG. 15), and/or a portion of the seventh strain gauge resistor (R₇) is positioned axially between the portions of the eighth strain gauge resistor (R₈). In some embodiments, one of the seventh and eighth strain gauge resistors (R₇, R₈) is a tension strain gauge resistor while the other is a compression strain gauge resistor.

As depicted in FIGS. 11B and 11C, in some embodiments, the fifth half-bridge circuit 3831E is positioned at the distal end portion 3815 of the beam 3810, while the sixth half-bridge circuit 3831F is positioned at the proximal end portion 3813 of the beam 3810. The fifth half-bridge circuit 3831E and the sixth half-bridge circuit 3831F can be electrically coupled to form a first secondary-full-bridge circuit. The first secondary-full-bridge circuit can be configured to measure strain imparted along the first axis. In some embodiments, the first secondary-full-bridge circuit can each be tension strain gauge resistors.

As depicted, the fifth half-bridge circuit 3831E can include the ninth strain gauge resistor (R₉) and the tenth strain gauge resistor (R₁₀). The ninth and tenth strain gauge resistors (R₉, R₁₀) can be positioned on opposite sides of the beam center axis A_{CL} and equidistant from the center axis. For example, the ninth and tenth strain gauge resistors (R₉, R₁₀) can be positioned equidistant between the beam center axis A_{CL} and a side edge of the surface to which they are mounted. In some embodiments, the ninth and tenth strain gauge resistors (R₉, R₁₀) can be positioned at the same distal position along the beam center axis A_{CL}. In some embodiments, the ninth and tenth strain gauge resistors (R₉, R₁₀) are both the same type of strain gauge resistor (e.g., are both tension strain gauge resistors). The fifth half-bridge circuit 3831E can be positioned longitudinally between the first half-bridge circuit 3831A and the second half-bridge circuit 3831B (e.g., proximally relative to the first half-bridge circuit 3831a and distally relative to the second half-bridge circuit 3831B).

As further depicted, the sixth half-bridge circuit 3831F can include the eleventh strain gauge resistor (R₁₁) and the twelfth strain gauge resistor (R₁₂). The eleventh and twelfth strain gauge resistors (R₁₁, R₁₂) can be positioned on opposite sides of the beam center axis A_{CL} and equidistant from the center axis. For example, the eleventh and twelfth strain gauge resistors (R₁₁, R₁₂) can be positioned equidistant between the beam center axis A_{CL} and a side edge of the surface to which they are mounted. In some embodiments, the eleventh and twelfth strain gauge resistors (R₁₁, R₁₂) can be positioned at the same proximal position along the beam center axis A_{CL}. In some embodiments, the eleventh and twelfth strain gauge resistors (R₁₁, R₁₂) are both the same type of strain gauge resistor (e.g., are both tension strain gauge resistors). The sixth half-bridge circuit 3831F can be positioned proximally relative to the second half-bridge circuit 3831B.

As depicted in FIGS. 11B and 11C, in some embodiments, the seventh half-bridge circuit 3831G is positioned at the distal end portion 3815 of the beam 3810, while the eighth half-bridge circuit 3831H is positioned at the proximal end portion 3813 of the beam 3810. The seventh half-bridge circuit 3831G and the eighth half-bridge circuit 3831H can be electrically coupled to form a second secondary-full-bridge circuit. The second secondary-full-bridge circuit can be configured to measure strain imparted along the second axis. In some embodiments, the second secondary-full-bridge circuit can be a combination of tension strain gauge resistors and compression strain gauge resistors.

As depicted, the seventh half-bridge circuit 3831G can include the thirteenth strain gauge resistor (R₁₃) and the fourteenth strain gauge resistor (R₁₄). The thirteenth and fourteenth strain gauge resistors (R₁₃, R₁₄) are positioned in axial alignment with the beam center axis A_{CL}. In some embodiments, a portion of the thirteenth strain gauge resistor (R₁₃) is positioned axially between the portions of the fourteenth strain gauge resistor (R₁₄), and/or a portion of the fourteenth strain gauge resistor (R₁₄) is positioned axially between the portions of the thirteenth strain gauge resistor (R₁₃). In some embodiments, one of the thirteenth and fourteenth strain gauge resistors (R₁₃, R₁₄) can be a tension strain gauge resistor while the other is a compression strain gauge resistor. The seventh half-bridge circuit 3831G can be positioned distally relative to the fourth half-bridge circuit 3831D. In some embodiments, the seventh half-bridge circuit 3831G can be positioned proximally relative to the third half-bridge circuit 3831C.

As further depicted, the eighth half-bridge circuit 3831H can include the fifteenth strain gauge resistor (R₁₅) and the sixteenth strain gauge resistor (R₁₆). The fifteenth and sixteenth strain gauge resistors (R₁₅, R₁₆) are positioned in axial alignment with the beam center axis A_{CL}. In some embodiments, a portion of the fifteenth strain gauge resistor (R₁₅) is positioned axially between the portions of the sixteenth strain gauge resistor (R₁₆), and/or a portion of the sixteenth strain gauge resistor (R₁₆) is positioned axially between the portions of the fifteenth strain gauge resistor (R₁₅) (e.g., as illustrated in FIG. 15). In some embodiments, one of the fifteenth and sixteenth strain gauge resistors (R₁₅, R₁₆) is a tension strain gauge resistor while the other is a compression strain gauge resistor. The eighth half-bridge circuit 3831H can be positioned proximally relative to the third half-bridge circuit 3831C.

In some embodiments, an output of the first secondary-full-bridge circuit can be redundant to a corresponding output of the first primary-full-bridge circuit. Similarly, an output of the second secondary-full-bridge circuit can be redundant to a corresponding output of the second primary-full-bridge circuit. In other words, absent a sensor malfunction, the respective outputs of the first and second secondary-full-bridge circuits substantially equal the respective outputs of the corresponding first and second primary-full-bridge circuits.

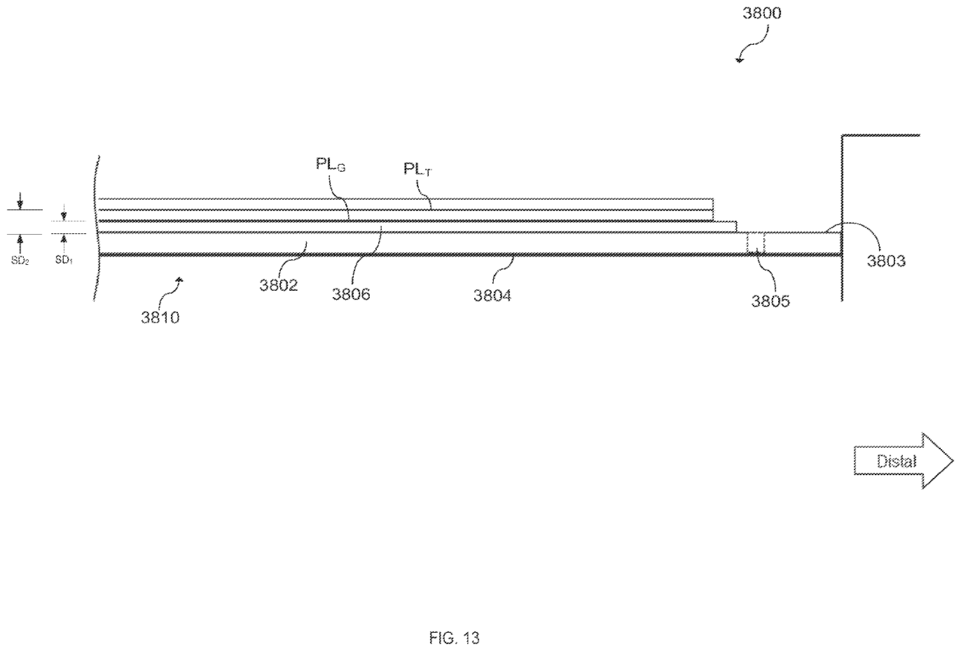

FIG. 12 is a perspective view of a proximal portion of the force sensor unit 3800, and FIG. 13 is a schematic side view of the portion of the force sensor unit 3800 depicted in FIG. 12. In so far as during certain operations, the beam 3810 can be capacitively coupled to the strain sensor 3830 when exposed to an electrical field, the orthogonal distance between the lateral surface 3812 of the beam 3810 and the strain gauges 3833 of the strain sensor 3830 can affect a current induced in the strain gauges 3833. When the distance between the strain gauges 3833 and the lateral surface 3812 is uniform, the induced current in each of the strain gauges 3833 is substantially equal to the induced current in each other strain gauge 3833. This equalization of the induced currents results in the canceling out of the effects of the electromagnetic interference in the output of the strain sensor 3830. In other words, since the induced current in each of the strain gauges 3833 has substantially the same value, the voltage of the output signals may have a greater magnitude, but the increase in voltage magnitude does not affect the voltage differential, and thus the indications of strain. However, variations in the flatness of the lateral surface 3812 and/or the thickness of an adhesive that couples the strain gauges 3833 to the beam 3810, can result in a lack of uniformity in the distance between the strain gauges 3833 and the lateral surface 3812 and corresponding variations in the induced currents, which, in turn, manifest in the output signals of the strain sensor 3830 as electromagnetic interference. As such, the force sensor unit 3800 disclosed herein, in some embodiments, utilizes an electrically conductive layer 3802 and an electrically insulative layer 3806 to facilitate uniform capacitive coupling when the force sensor unit 3800 is exposed to an electrical field.

As depicted in FIG. 12, the force sensor unit 3800 includes the electrically conductive layer 3802 over the lateral surface 3812 of the beam 3810. In some embodiments, the electrically conductive layer 3802 is mechanically bonded to the lateral surface 3812 of the beam 3810 along the length (e.g., a longitudinal length) of the electrically conductive layer 3802. In addition to the mechanical bonding, the electrically conductive layer 3802 is also electrically coupled to the beam 3810. In such an embodiments, the mechanical bonding facilitates the accurate measurement of strain resulting from the deflection of the beam 3810, while the electrical coupling transfers an electrical current from the beam 3810 to the electrically conductive layer 3802. As a result, the current conducted by the electrically conductive layer 3802 can cause the induced currents in the strain sensor 3830 instead of the induced current resulting from the current in the beam 3810. As such, the magnitude of the induced current is dependent, at least in part, on the separation distance (e.g., separation distance SD₁ and/or separation distance SD₂) between the components of the strain sensor 3830 and the electrically conductive layer 3802 rather than the distance between the components and the lateral surface 3812. In other words, with regards to capacitive coupling, the electrically conductive layer 3802 replaces the lateral surface 3812 of the beam 3810 due to the electrical coupling therebetween.

As depicted in FIGS. 10 and 12, in order to electrically couple the electrically conductive layer 3802 to the lateral surface 3812, in some embodiments, the electrically conductive layer 3802 includes two or more weld locations 3805. The electrically conductive layer 3802 is electrically coupled to the beam 3810 at each of the weld locations 3805, such as via a weld. In some embodiments, the weld locations 3805 are positioned to produce a spatially uniform electrical coupling of the electrically conductive layer 3802 to the beam 3810. For example, as depicted in FIG. 10, in some embodiments, the force sensor unit 3800 has eight weld locations 3805 that are distributed around a perimeter of the electrically conductive layer 3802. However, in additional embodiments, the force sensor unit 3800 can include three, four, six, or ten weld locations 3805 distributed about the electrically conductive layer 3802. The distribution of the weld locations 3805 can facilitate a substantially uniform resistance to the current from the beam 3810 along the longitudinal length of the electrically conductive layer 3802 and across the lateral width of the electrically conductive layer 3802.

In some embodiments, the electrically conductive layer 3802 is mechanically bonded to the lateral surface 3812 of the beam 3810 via an adhesive. For example, the electrically conductive layer 3802 can be mechanically bonded to the lateral surface 3812 via an epoxy resin, an ethyl-based cyanoacrylate glue, a methyl-based cyanoacrylate glue, a phenolic resin, or other suitable adhesive. In some embodiments, the adhesive is distributed laterally (e.g., parallel to the lateral axis A_{LA}) across the lateral surface 3812 of the beam 3810 and longitudinally (e.g., parallel to the longitudinal axis A_{LO}) along the lateral surface 3812 of the beam 3810. The adhesive can, for example, cover at least 90 percent (e.g., at least 95 percent) of a surface 3804 of the electrically conductive layer 3802 that faces the beam 3810.

In some embodiments, the electrically conductive layer 3802 can have a top surface 3803 facing the strain sensor 3830 that is within a specified flatness tolerance. The flatness tolerance defines a maximal separation distance between a plane passing through the highest point of the surface and a parallel plane passing through the lowest point of the surface. The specified flatness tolerance can, for example, be 0.1 micrometers or less. The flatness tolerance of the top surface 3803 of the electrically conductive layer 3802 facilitates the positioning of the strain sensor 3830 at a uniform separation distance SD₁ from the electrically conductive layer 3802. Similarly, in some embodiments, the top surface 3803 of the electrically conductive layer 3802 can have a surface roughness that is less than 0.1 micrometers to facilitate the uniform separation distance SD₁. In some embodiments, the electrically conductive layer 3802 can have a thickness TCL that is within a specified thickness range. The specified thickness range can, for example, be greater than 45 micrometers and less than or equal to 55 micrometers. The electrically conductive layer 3802 being within thickness range can facilitate a uniformity of stiffness and a uniformity of electrical resistance within the electrically conductive layer 3802.

In some embodiments, the electrically insulative layer 3806 is positioned over the electrically conductive layer 3802. The strain sensor 3830 is positioned over (e.g., formed on) a length of the electrically insulative layer 3806. In other words, the strain sensor 3830 is physically separated from the electrically conductive layer 3802 by the electrically insulative layer 3806, which precludes the establishment of a physical electrical connection between the strain sensor 3830 and the electrically conductive layer 3802. In other words, the electrically insulative layer 3806 precludes a conductive electrical coupling between the strain sensor 3830 and the electrically conductive layer 3802 and, thus, the beam 3810. In some embodiments, the electrically insulative layer 3806 has a uniform thickness. The uniform thickness of the electrically insulative layer 3806 establishes the uniform separation distance SD₁ between the strain sensor 3830 and the electrically conductive layer 3802. For example, in some embodiments, an electrically insulative layer 3806 of the uniform thickness is positioned over an electrically conductive layer 3802 that has a specified flatness in order to position each strain gauge 3833 of the strain sensor 3830 at an equal distance (i.e., the uniform separation distance SD₁) from the electrically conductive layer 3802.

In some embodiments, the electrically conductive layer 3802 can, for example, be a stainless steel, such as grade 304 austenitic stainless steel, grade 316 austenitic stainless steel, or other suitable stainless steel alloy. In some embodiments, the electrically insulative layer 3806 can, for example, be a polyamide film or other suitable electrically insulative film.

Referring again to FIGS. 10-13, the strain sensor 3830 includes a bridge circuit 3831, a set of electrical pads 3839 (e.g., contacts, tap points, or pickup points), and an electrical trace structure 3820. As depicted, the bridge circuit 3831 (e.g., a set of split half-bridge circuits distributed along the longitudinal axis A_{LO} of the beam 3810) includes a set of strain gauges 3833 that are over (e.g., formed on) the electrically insulative layer 3806. The bridge circuit 3831 has a uniform separation distance SD₁ from the electrically conductive layer 3802. The electrical trace structure 3820 is electrically coupled between the sensor cable 3840 and the electrical pads 3839. As such, the electrical trace structure 3820 can provide an input voltage to the bridge circuit 3831 and can transmit an output signal indicative of strain to the sensor cable 3840.

In some embodiments, the strain gauges 3833 are positioned on a gauge plane PL_{G} (e.g., in a gauge layer) that is parallel to the electrically conductive layer 3802. Being parallel with the electrically conductive layer 3802 the gauge plane PL_{G} is separated from the electrically conductive layer 3802 by the uniform separation distance SD₁. The electrical trace structure 3820 is similarly positioned on a trace plane PL_{T} (e.g., in a trace layer). The trace plane PL_{T} is parallel to the gauge plane PL_{G}. The trace plane PL_{T} is separated from the electrically conductive layer 3802 by a uniform separation distance SD₂. In some embodiments, the distance between the trace plane PL_{T} and the electrically conductive layer 3802 is greater than the distance between the gauge plane PL_{G} and the electrically conductive layer 3802. The electrical trace structure 3820 is laterally offset from each strain gauge 3833. This positioning of the electrical trace structure 3820 facilitates a uniformity in a stiffness of the strain sensor 3830 that extends parallel to the lateral surface 3812 of the beam 3810. The uniformity of the stiffness of the strain sensor 3830 facilitates the accurate measurement of the strain developed in the beam 3810 in response to a load applied to the instrument. In contrast, localized stiffness concentrations resulting from the stacking or overlapping of components, such as the positioning of the electrical trace structure 3820 at the same lateral and longitudinal point as a strain gauges 3833 can establish a localized stiffness concentration. The localized stiffness concentration can affect the response of the co-located strain gauge 3833 to the deflection of the beam 3810 and, thus, the magnitude of the strain indicated by the strain gauge 3833.

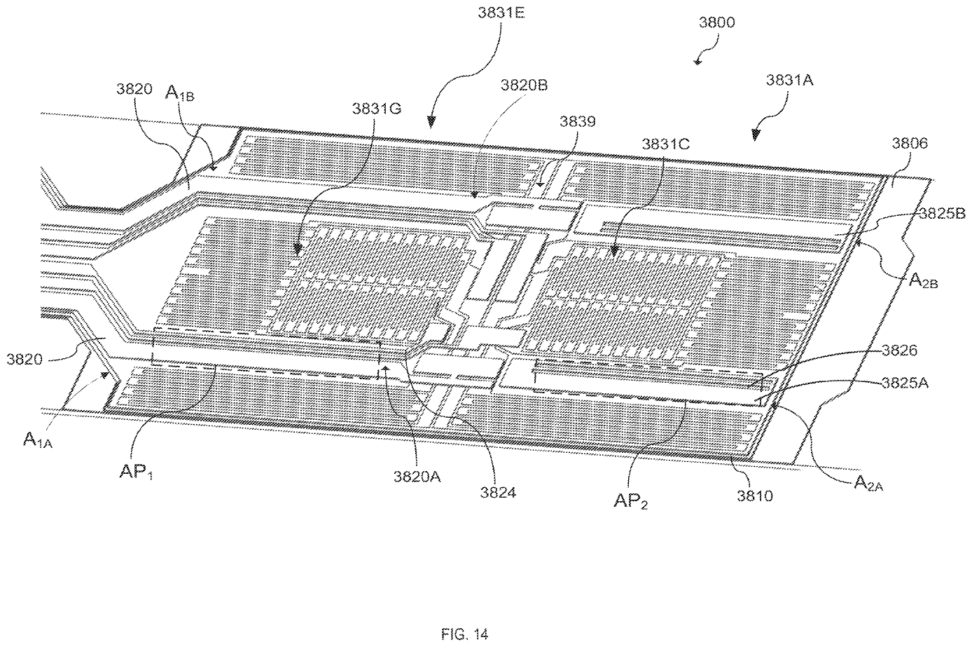

Referring now to FIGS. 10, 11A, and 14, a perspective view of a distal portion of the force sensor unit 3800 is depicted in FIG. 14. While the distal portion of the strain sensor 3830 is depicted in FIG. 14, the strain sensor 3830 also includes a proximal portion that substantially mirrors the depicted distal portion. In order to mitigate the potential electromagnetic interference stemming from inductive coupling, in some embodiments, the force sensor unit 3800 includes the strain sensor 3830 mechanically coupled to the beam 3810. The strain sensor includes a first region A₁ and a second region A₂. The strain sensor 3830 also includes the bridge circuit 3831, the electrical trace structure 3820 and a balancing structure 3825. The electrical trace structure 3820 is positioned over the first region A₁ and is electrically coupled to the bridge circuit 3831. The balancing structure 3825 is positioned over the second region A₂ and has an absence of physical electrical connections with any other component of the force sensor unit 3800. In other words, the balancing structure 3825 can be a conductive component that does not form a conductive path with any other conductive component of the force sensor unit 3800. In some embodiments, the balancing structure 3825 can be a conductive copper trace that is completely surrounded by an insulating material. For example, the balancing structure 3825 can be formed as a trace structure that is not in physical contact with any other electrically conductive component of the force sensor unit 3800.

As depicted in FIG. 14, in some embodiments, the electrical trace structure 3820 includes a first area portion AP₁. Similarly, the balancing structure 3825 includes a second area portion AP₂. An outline of the first area portion AP₁ of the electrical trace structure 3820 defines a first pattern. The first pattern has a first surface area. The outline of the first area portion AP₁ can, for example, be defined by each edge (e.g., perimeter edges) of the portions of electrically conductive traces (e.g., deposited copper traces) positioned within the first area portion AP₁. Accordingly, the first surface area can include the combined surface area of the portions of the electrically conductive traces within the first area portion AP₁. An outline of the second area portion AP₂ of the balancing structure 3825 defines a second pattern. The second pattern as a second surface area. The outline of the second area portion AP₂ can, for example, be defined by a combination of the edges of the conductive material (e.g., copper) that are in contact with an insulative material of the trace layer PL_{T} within the second area portion AP₂. Accordingly, the second surface area can include the surface area of the conductive material that is within the second area portion AP₂.

In some embodiments, the second pattern of the balancing structure 3825 matches the first pattern of the electrical trace structure 3820. Similarly, the second surface area of the balancing structure 3825 is substantially equal to the first surface area of the electrical trace structure 3820. The second pattern of the balancing structure 3825 can, for example, be configured to generate a first voltage change in the strain sensor 3830 that is proportional to a second voltage change generated in the strain sensor 3830 by the electrical trace structure 3820. In other words, the second pattern can be configured such that the balancing structure 3825 has substantially the same inductive coupling with a first adjacent component that the electrical trace structure 3820 has with a second adjacent component. In some embodiments, a longitudinal axis of the second pattern of the balancing structure 3825 is aligned with a longitudinal axis of the first pattern of the electrical trace structure 3820. In other words, the second pattern and the first pattern can be located at the same lateral position and can extend parallel to strain gauges 3833 of the strain sensor 3830. It should be appreciated that the establishment of symmetry between the first pattern and the second pattern and the first surface area and the second surface area facilitates the mitigation of the electromagnetic interference by providing substantially equal induced currents to the positive and negative traces of the strain sensor 3830.

In some embodiments, the electrical trace structure 3820 includes an input trace 3822. The electrical trace structure 3820 can also include one or more measurement traces 3824 (e.g., signal traces). The input trace 3822 is configured to deliver an input voltage (e.g., an excitation voltage) from the sensor cable 3840 to one or more split half-bridge circuits 3831. The measurement trace 3824 is configured to deliver an output signal from the split half-bridge circuit 3831 to the sensor cable 3840. As depicted, the input trace 3822 can have a lateral width that is greater than a lateral width of the measurement trace 3824 when oriented parallel to the longitudinal axis A_{LO}. The first area portion AP₁ of the electrical trace structure 3820 includes a portion of the input trace 3822 and a portion of the measurement trace 3824. In some embodiments, the second pattern of the balancing structure 3825 defines a void 3826 that corresponds to the separation between the input trace 3822 and the measurement trace 3824.

In some embodiments wherein the half-bridge circuit 3831 are arranged as the four bridge-circuit combinations 3832, 3834, 3836, 3838, the strain sensor 3830 can include four electrical trace structures 3820 and four balancing structures 3825. Each of the electrical trace structures 3820 and the balancing structures 3825 can be positioned on the trace plane PL_{T} (e.g., within a single trace layer) that is over and parallel to the gauge plane PL_{G}. Each of the electrical trace structures 3820 and the balancing structures 3825 is laterally offset from each strain gauge 3833. This lateral offset facilitates a uniformity in the stiffness (e.g., rigidity) of the strain sensor. In other words, offsetting the electrical trace structures 3820 and the balancing structures 3825 from the strain gauges 3833 precludes the generation of stiffness concentrations at the points of overlap between the strain gauges 3833 and the electrical trace structures 3820 and/or the balancing structures 3825.

In some embodiments, the beam 3810 includes a beam center axis A_{CL} that is along the lateral face 3812 and parallel to the longitudinal axis A_{LO}. The four bridge-circuit combinations 3832, 3834, 3836, 3838 can be arranged along the beam center axis A_{CL}. For example, in some embodiments, the primary distal bridge-circuit combination 3832 is distal of the secondary distal bridge-circuit combination 3836, and the primary proximal bridge-circuit combination 3834 is proximal of the secondary distal bridge-circuit combination 3836 and distal of the secondary proximal bridge-circuit combination 3838. In such an embodiment, the first regions (A_{1A}, A_{1B}), a first electrical trace structure 3820A and a second electrical trace structure 3820B of the four electrical trace structures 3820 are positioned on opposite sides of the beam center axis A_{CL}, equidistant from the beam center axis A_{CL}, and adjacent to the secondary distal bridge-circuit combination 3836. Similarly, a third electrical trace structure and a fourth electrical trace structure of the four electrical trace structures 3820 are positioned on opposite sides of the beam center axis A_{CL}, equidistant from the beam center axis A_{CL}, and adjacent to the primary proximal bridge-circuit combination 3836. A first balancing structure 3825A of the four balancing structures 3825 is positioned proximal to the first electrical trace structure, in alignment with the first electrical trace structure, and adjacent to the primary distal bridge-circuit combination 3832. Similarly, a second balancing structure 3825B is positioned proximal to the second electrical trace structure, in alignment with the second electrical trace structure 3820B, and adjacent to the primary distal bridge-circuit combination 3832. Additionally, a third balancing structure of the four balancing structures 3825 is positioned proximal to the third electrical trace structure, in alignment with the third electrical trace structure, and adjacent to the secondary proximal bridge-circuit combination 3838. Further, in some embodiments, a fourth balancing structure of the four balancing structures 3825 is positioned proximal to the fourth electrical trace structure, in alignment with the fourth electrical trace structure, and adjacent to the secondary proximal bridge-circuit combination 3838. It should be appreciated that as described, the arrangement of the electrical trace structures 3820 and the balancing structures 3825 maximizes the degree of symmetry of the strain sensor 3830 about the longitudinal axis A_{LO} and/or about the lateral axis A_{LA}. This symmetry facilitates the mitigation of the effects of electromagnetic interference and the uniformity in the stiffness of the strain sensor.

FIG. 15 depicts a portion of a force sensor unit 4800 for use with a force sensing medical instrument, such as instrument 1400 described herein. In some embodiments, the force sensor unit 4800 or any of the components therein are optionally parts of a surgical system that performs surgical procedures. The surgical system may include a manipulator unit, a series of kinematic linkages, a series of cannulas, or the like. The force sensor unit 4800 (and any of the force sensor units described herein) can be used in any suitable surgical system, such as the MIRS system 1000 shown and described above to mitigate the effects of electromagnetic interference when the instrument is exposed to an electrical field. In some embodiments, the force sensor unit 4800 can include any of the components or features disclosed herein with reference to force sensor unit 1800, force sensor unit 2800, and/or force sensor unit 3800.

FIG. 15 illustrates an arrangement of the strain gauge resistors of a strain sensor 4830 according to that depicted with reference to strain sensor 3830 of FIGS. 10 and 11B-11C. FIG. 15 depicts the four half-bridge circuits of first and second primary-full-bridge circuits and first and second secondary-full-bridge circuits of the strain sensor 4830 configured to be positioned at the proximal end portion of the beam (e.g., the beam 3810). As illustrated, the proximal end portion of the strain sensor 4830 can include the half-bridge-circuits 4831B and 4831D of the first and second primary-full-bridge circuits, such as described with reference to FIGS. 11B and 11C, and the half-bridge-circuits 4831F and 4831H of the first and second secondary-full-bridge circuits, such as described with reference to FIGS. 11B and 11C. Said another way, the proximal end portion of the strain sensor 4830 can include the four proximal end portion half-bridge circuits 4831 with the substantially similar corresponding distal end portion half-bridge circuits of the first and second primary-full-bridge-circuits and the first and second secondary-full-bridge-circuits not being depicted in FIG. 15. As previously described, the strain sensor 4830 is positioned over an electrically insulative layer 4806 that is positioned over an electrically conductive layer 4802. The electrically conductive layer 4802 is electrically coupled to the beam 4810 at a number of weld locations 4805.

As further depicted in FIG. 15, in some embodiments, the strain sensor 4830 can include a balancing structure 4825. The balancing structure 4825 can include any of the features and/or elements described herein with reference to balancing structures 2825 and 3825. The balancing structure 4825 has an absence of physical electrical connections with any other component of the strain sensor 4830. In other words, the balancing structure 4825 can be a conductive component that does not form a conductive path with any other conductive component of the strain sensor 4830. In some embodiments, the balancing structure 4825 can be a conductive copper trace that is completely surrounded by an insulating material. For example, the balancing structure 4825 can be formed as a trace structure that is not in physical contact with any other electrically conductive component of the strain sensor 4830.

As depicted in FIG. 15, in reference to the proximal end portion half-bridge-circuit 4831B of the first primary full-bridge circuit, the strain gauge resistors R₃, R₄ are configured to be positioned on opposite sides of the beam center axis A_{CL} and equidistant from the center axis. For example, the strain gauge resistors R₃, R₄ can be positioned equidistant between the beam center axis A_{CL} and a side edge of the surface to which they are mounted. In some embodiments, the strain gauge resistors R₃, R₄ can be positioned at the same proximal position along the beam center axis A_{CL}. As depicted, in some embodiments, the strain gauge resistors R₃, R₄ are both the same type of strain gauge resistor (e.g., are both tension strain gauge resistors). As further depicted in FIG. 15, strain gauge resistors R₇, R₈ of the proximal end portion half-bridge circuit 4831D of the second primary full-bridge circuit are configured to be positioned in axial alignment with the beam center axis A_{CL}. As depicted, strain gauge resistor R₈ is positioned between the portions of strain gauge resistor R₇. As depicted, strain gauge resistors R₇ is a tension strain gauge resistor while strain gauge resistor R₈ is a compression strain gauge resistor. As further depicted in FIG. 15, proximal end portion strain gauge resistors R₁₁, R₁₂, R₁₅, and R₁₆ of the half-bridge circuits 4831F and 4831H of the first and second secondary-full-bridge circuits can be positioned in a similar arrangement. Further, the distal end portion half-bridge circuits of the first and second primary-full-bridge circuits and the first and second secondary-full-bridge circuits are not depicted as noted above, but can be arranged in a similar manner (e.g., mirroring that of the proximal end portion half-bridge circuits).

As depicted in FIG. 15, in some embodiments, the bridge circuit 4831 can be surrounded by a wall 4807. In other words, the wall 4807 can form a perimeter around portions of the strain sensor 4830. The wall 4807 can be positioned on the gauge plane and can extend away from the beam 4810. The wall 4807 can have a height above the gauge plane that is equal to or greater than a thickness of the strain gauges. The wall 4807 can, for example, be formed from a copper-nickel alloy wire. The presence of the wall 4807 can form a barrier to liquid intrusion, thereby increasing a durability of the strain sensor 4830. For example, the wall 4807 can shield the strain gauges from direct contact with high-temperature cleaning fluids (e.g., steam under pressure) during post-procedure processing (e.g., autoclaving).

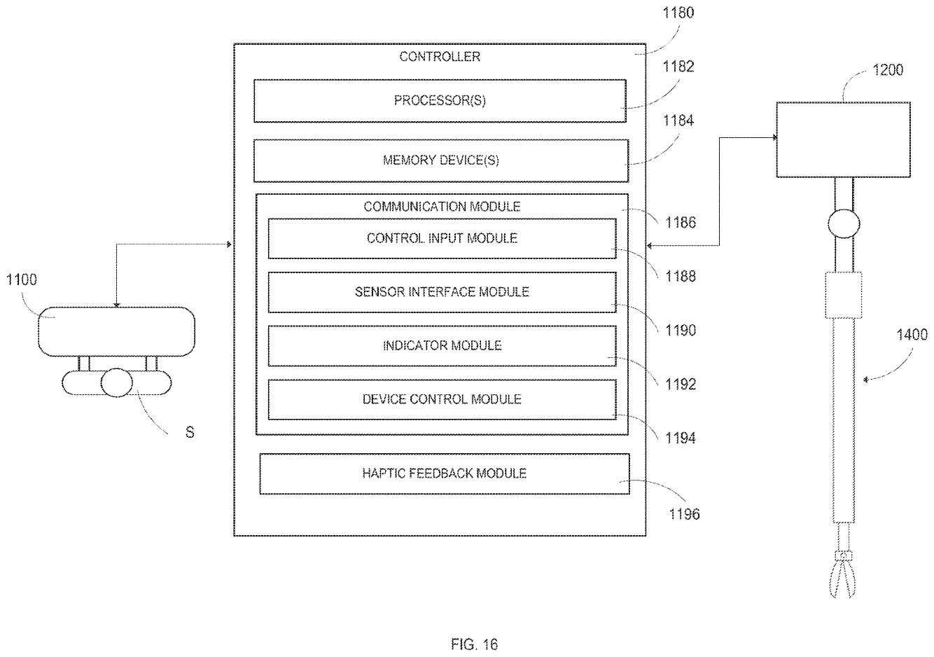

As shown particularly in FIG. 16, a schematic diagram of one embodiment of suitable components that may be included within the controller 1180 is illustrated. In some embodiments, the controller 1180 is positioned within a component of the surgical system 1000, such as the user control unit 1100 and/or the optional auxiliary equipment unit 1150. However, the controller 1180 may also include distributed computing systems wherein at least one aspect of the controller 1180 is at a location which differs from the remaining components of the surgical system 1000 for example, at least a portion of the controller 1180 may be an online controller.

As depicted, the controller 1180 includes one or more processor(s) 1182 and associated memory device(s) 1184 configured to perform a variety of computer implemented functions (e.g., performing the methods, steps, calculations and the like and storing relevant data as disclosed herein). Additionally, in some embodiments, the controller 1180 includes a communication module 1186 to facilitate communications between the controller 1180 and the various components of the surgical system 1000.

As used herein, the term “processor” refers not only to integrated circuits referred to in the art as being included in a computer, but also refers to a controller, a microcontroller, a microcomputer, a programmable logic controller (PLC), an application specific integrated circuit, and other programmable circuits. Additionally, the memory device(s) 1184 may generally comprise memory element(s) including, but not limited to, computer readable medium (e.g., random access memory (RAM)), computer readable nonvolatile medium (e.g., a flash memory), a floppy disc, a compact disc read only memory (CD ROM), a magneto optical disc (MOD), a digital versatile disc (DVD) and/or other suitable memory elements. Such memory device(s) 1184 may generally be configured to store suitable computer readable instructions that, when implemented by the processor(s) 1182, configure the controller 1180 to perform various functions.

In some embodiments, the controller 1180 includes a haptic feedback module 1196. The haptic feedback module 1196 may be configured to deliver a haptic feedback to the operator based on inputs received from a force sensor unit 1180 of the instrument 1400. In some embodiments, haptic feedback module 1196 may be an independent module of the controller 1180. However, in some embodiments the haptic feedback module 1196 may be included within the memory device(s) 1184.

The communication module 1186 may include a control input module 1188 configured to receive control inputs from the operator/surgeon S, such as via the input device 1116 of the user control unit 1100. The communication module may also include an indicator module 1192 configured to generate various indications in order to alert the operator.

The communication module 1186 may also include a sensor interface 1190 (e.g., one or more analog to digital converters) to permit signals transmitted from one or more sensors (e.g., strain sensors of the force sensor unit 1180) to be converted into signals that can be understood and processed by the processors 1182. The sensors may be communicatively coupled to the communication module 1186 using any suitable means. For example the sensors may be coupled to the communication module 1186 via a wired connection and/or via a wireless connection, such as by using any suitable wireless communications protocol known in the art. Additionally, in some embodiments, the communication module 1186 includes a device control module 1814 configured to modify an operating state of the instrument 1400 (and/or any of the instruments described herein. Accordingly, the communication module is communicatively coupled to the manipulator unit 1200 and/or the instrument 1400. For example, the communications module 1186 may communicate to the manipulator unit 1200 and/or the instrument 1400 an excitation voltage for the strain sensor(s), a handshake and/or excitation voltage for a positional sensor (e.g., for detecting the position of the designated portion relative to the cannula), cautery controls, positional setpoints, and/or an end effector operational setpoint (e.g., gripping, cutting, and/or other similar operation performed by the end effector).

While various embodiments have been described above, it should be understood that they have been presented by way of example only, and not limitation. Where methods and/or schematics described above indicate certain events and/or flow patterns occurring in certain order, the ordering of certain events and/or operations may be modified. While the embodiments have been particularly shown and described, it will be understood that various changes in form and details may be made.

For example, any of the instruments described herein (and the components therein) are optionally parts of a surgical assembly that performs minimally invasive surgical procedures, and which can include a manipulator unit, a series of kinematic linkages, a set of cannulas, or the like. Thus, any of the instruments described herein can be used in any suitable surgical system, such as the MIRS system 1000 shown and described above. Moreover, any of the instruments shown and described herein can be used to manipulate target tissue during a surgical procedure. Such target tissue can be cancer cells, tumor cells, lesions, vascular occlusions, thrombosis, calculi, uterine fibroids, bone metastases, adenomyosis, or any other bodily tissue. The presented examples of target tissue are not an exhaustive list. Moreover, a target structure can also include an artificial substance (or non-tissue) within or associated with a body, such as for example, a stent, a portion of an artificial tube, a fastener within the body or the like.

For example, any of the components of a surgical instrument as described herein can be constructed from any material, such as medical grade stainless steel, nickel alloys, titanium alloys or the like. Further, any of the links, tool members, beams, shafts, cables, or other components described herein can be constructed from multiple pieces that are later joined together. For example, in some embodiments, a link can be constructed by joining together separately constructed components. In other embodiments, however, any of the links, tool members, beams, shafts, cables, or components described herein can be monolithically constructed.

For example, any of the strain sensor configurations described or contemplated herein (e.g., as depicted in FIGS. 11A-11C) can include any of the strain gauge resistor arrangements described or contemplated herein (e.g., as depicted in FIGS. 12, 14, and 15).

Although various embodiments have been described as having particular features and/or combinations of components, other embodiments are possible having a combination of any features and/or components from any of embodiments as discussed above. Aspects have been described in the general context of medical devices, and more specifically surgical instruments, but inventive aspects are not necessarily limited to use in medical devices.

1. A force sensor unit, comprising:
a beam including a lateral surface;
an electrically conductive layer over the lateral surface;
an electrically insulative layer over the electrically conductive layer; and
a strain sensor over a length of the electrically insulative layer;
wherein the electrically conductive layer is mechanically bonded to the lateral surface of the beam along a length of the electrically conductive layer; and
wherein the electrically conductive layer is electrically coupled to the beam.

2. The force sensor unit of claim 1, wherein:
the electrically conductive layer has a top surface facing the strain sensor; and
the top surface is within a specified flatness tolerance.

3. The force sensor unit of claim 1, wherein:
the electrically conductive layer includes two or more weld locations; and
the electrically conductive layer is electrically coupled to the beam at each of the weld locations.

4. The force sensor unit of claim 3, wherein:
the two or more weld locations are positioned to produce a spatially uniform electrical coupling of the electrically conductive layer to the beam.

5. (canceled)

6. The force sensor unit of claim 1, wherein:
the electrically conductive layer is mechanically bonded to the lateral surface of the beam along the length of the electrically conductive layer via an adhesive;
the adhesive is distributed laterally across the lateral surface of the beam and longitudinally along the lateral surface of the beam; and
the adhesive covers at least 95 percent of a surface of the electrically conductive layer facing the beam.

7. The force sensor unit of claim 1, wherein:
the electrically insulative layer has a uniform thickness; and
the uniform thickness establishes a uniform separation distance between the strain sensor and the electrically conductive layer.

8. The force sensor unit of claim 2, wherein:
the specified flatness tolerance is 0.1 micrometers or less.

9. The force sensor unit of claim 1, wherein:
the electrically conductive layer has a thickness that is within a specified thickness range; and
the specified thickness range is greater than 45 micrometers and less than 55 micrometers.

10. The force sensor unit of claim 1, wherein:
the electrically conductive layer has a surface roughness of less than 0.1 micrometers.

11. The force sensor unit of claim 1, wherein:
the strain sensor includes a bridge circuit, a plurality of electrical pads, and an electrical trace structure;
the strain sensor has a stiffness extending parallel to the lateral surface of the beam;
the bridge circuit includes a plurality of strain gauges formed over the electrically insulative layer;
the bridge circuit has a uniform separation distance from the electrically conductive layer;
the electrical trace structure is electrically coupled to the plurality of electrical pads;
the plurality of strain gauges is positioned on a gauge plane that is parallel to the electrically conductive layer;
the electrical trace structure is positioned on a trace plane that is parallel to the gauge plane; and
the electrical trace structure is laterally offset from each strain gauge of the plurality of strain gauges to facilitate a uniformity in the stiffness of the strain sensor.

12. The force sensor unit of claim 1, wherein:
the strain sensor has a stiffness extending parallel to the lateral surface;
the force sensor unit includes an enclosure layer;
the enclosure layer covers the strain sensor;
the enclosure layer has a uniform thickness; and
the uniform thickness facilitates a uniformity in the stiffness of the strain sensor.

13. The force sensor unit of claim 1, wherein:
the strain sensor includes a bridge circuit;
the bridge circuit includes a plurality of strain gauges formed over the electrically insulative layer;
a wall surrounds the bridge circuit; and
the wall has a height that is equal to or greater than a thickness of the strain gauges.

14. A force sensor unit, comprising:
a beam; and
a strain sensor mechanically coupled to the beam and including a first region, a second region, a bridge circuit, an electrical trace structure, and a balancing structure;
wherein the electrical trace structure is over the first region;
wherein the balancing structure is over the second region;
wherein the electrical trace structure is electrically coupled to the bridge circuit; and
wherein the balancing structure has an absence of physical electrical connections with any other component of the force sensor unit.

15. The force sensor unit of claim 14, wherein:
the electrical trace structure includes a first area portion;
the balancing structure includes a second area portion;
an outline of the first area portion of the electrical trace structure defines a first pattern having a first surface area;
an outline of the second area portion of the balancing structure defines a second pattern having a second surface area;
the second pattern of the balancing structure matches the first pattern of the electrical trace structure; and
the second surface area of the balancing structure equals the first surface area.

16. The force sensor unit of claim 15, wherein:
the second pattern of the balancing structure is configured to generate a first voltage change in the strain sensor that is proportional to a second voltage change generated in the strain sensor by the electrical trace structure.

17. The force sensor unit of claim 15, wherein:
the electrical trace structure includes an input trace separated from one or more measurement traces;
the first area portion of the electrical trace structure includes a portion of the input trace and a portion of the one or more measurement traces; and
the second pattern defines a void that corresponds to the separation between the input trace and the one or more measurement traces.

18. The force sensor unit of claim 15, wherein:
a longitudinal axis of the second pattern of the balancing structure is aligned with a longitudinal axis of the first pattern of the electrical trace structure.

19. The force sensor unit of claim 14, wherein:
the force sensor unit includes an electrically conductive layer and an electrically insulative layer;
the beam includes a lateral surface;
the electrically conductive layer is over the lateral surface;
the electrically insulative layer is over the electrically conductive layer;
the strain sensor is over a length of the electrically insulative layer;
the electrically conductive layer is mechanically bonded to the lateral surface of the beam along the length of the electrically conductive layer; and
the electrically conductive layer is electrically coupled to the beam.

20. The force sensor unit of claim 19, wherein:
the strain sensor has a stiffness extending parallel to the lateral surface of the beam;
the bridge circuit is one of eight half-bridge circuits;
each half-bridge circuit of the eight half-bridge circuits includes a plurality of strain gauges;
the eight half-bridge circuits are arranged as four full-bridge-circuit combinations;
the electrical trace structure is a first electrical trace structure of four electrical trace structures;
each of the four electrical trace structures includes an input trace separated from one or more measurement traces;
the balancing structure is one of four balancing structures;
each half-bridge circuit of the eight half-bridge circuits includes two strain gauges of the plurality of strain gauges formed over the electrically insulative layer;
each of the plurality of strain gauges is positioned within a gauge plane that is parallel to the electrically conductive layer and has a uniform separation distance from the electrically conductive layer;
each of the four electrical trace structures and each of the four balancing structures is positioned on a trace plane that is over and parallel to the gauge plane; and
each of the four electrical trace structures and each of the four balancing structures is laterally offset from each strain gauge of the plurality of strain gauges to facilitate a uniformity in the stiffness of the strain sensor.

21. The force sensor unit of claim 20, wherein:
the beam includes a beam center axis;
the four full-bridge-circuit combinations include a primary proximal bridge-circuit combination, a primary distal bridge-circuit combination, a secondary proximal bridge-circuit combination, and a secondary distal bridge-circuit combination;
the primary proximal bridge-circuit combination is proximal of the secondary proximal bridge-circuit combination;
the primary distal bridge-circuit combination is proximal of the secondary distal bridge-circuit combination and distal of the secondary proximal bridge-circuit combination;
a first electrical trace structure and a second electrical trace structure of the four electrical trace structures are positioned on opposite sides of the beam center axis, equidistant from the beam center axis, and adjacent to the secondary distal bridge-circuit combination;
a third electrical trace structure and a fourth electrical trace structure of the four electrical trace structures are positioned on opposite sides of the beam center axis, equidistant from the beam center axis, and adjacent to the primary proximal bridge-circuit combination;
a first balancing structure of the four balancing structures is positioned proximal to the first electrical trace structure, in alignment with the first electrical trace structure, and adjacent to the primary distal bridge-circuit combination;
a second balancing structure of the four balancing structures is positioned proximal to the second electrical trace structure, in alignment with the second electrical trace structure, and adjacent to the primary distal bridge-circuit combination;
a third balancing structure of the four balancing structures is positioned proximal to the third electrical trace structure, in alignment with the third electrical trace structure, and adjacent to the secondary proximal bridge-circuit combination; and
a fourth balancing structure of the four balancing structures is positioned proximal to the fourth electrical trace structure, in alignment with the fourth electrical trace structure, and adjacent to the secondary proximal bridge-circuit combination.

22-25. (canceled)
